(19) United States
(12) Patent Application Publication
(10) Pub. No.: US 2018/0092827 A1
(43) Pub. Date: Apr. 5, 2018

(54) ONE PART ACRYLIC NAIL FORMULATION WITH DISCONTINUOUS PHASE

(71) Applicants: MYCONE DENTAL SUPPLY CO., INC., Gibbstown, NJ (US); NAIL ALLIANCE, LLC, Gladstone, MO (US)

(72) Inventors: Kevin SHERAN, Haddonfield, NJ (US); Danny Lee HAILE, La Habra, CA (US)

(21) Appl. No.: 15/817,115

(22) Filed: Nov. 17, 2017

Related U.S. Application Data
(63) Continuation-in-part of application No. 15/452,532, filed on Mar. 7, 2017.
Application No. 15/452,532 is a continuation of application No. PCT/US16/37660, filed on Jun. 15, 2016.

(51) Int. Cl. A61K 8/87; A61Q 3/02; A61K 8/36; A61K 8/362; A61K 8/891; A61K 8/92; A45D 34/00

(52) U.S. Cl. CPC A61K 8/87; A61Q 3/02; A61K 8/36; A61K 8/362; A61K 2800/81; A61K 8/92; A45D 34/00; A61K 2800/412; A61K 2800/95; A61K 8/891

(57) ABSTRACT

A photopolymerizable composition for forming a cosmetic coating for nails comprises a continuous phase and a discontinuous phase. The continuous phase comprises (meth)acrylate monomers, crosslinkers, photoinitiators, and optionally (meth)acrylate oligomers and other additives. The discontinuous phase comprises particles such as polymers, inorganic materials or a mixture thereof. The particle sizes of the components of the discontinuous phase mixture are in the range of several microns or tens of microns. This composition has good storage stability, exhibits high viscosity, and has a relatively low exotherm when used. The composition can be shaped readily by a nail technician to form nail coatings and is then exposed to actinic light to form a nail coating. The photopolymerizable composition may be kept in a container such as a tube container, a syringe container, and a foil packet.

This application claims priority as a continuation in part application to U.S. patent application No. 15/452,532 filed on 7 Mar. 2017, entitled “One Part Acrylic Nail Formulation”, now pending, which in turn claims priority as a continuation of International Application PCT/US2016/037660, with an international filing date of 15 Jun. 2016, entitled “One Part Acrylic Nail Formulation”, now pending. Each of these applications is incorporated by reference in their entirety.

### FIELD OF THE INVENTION

The present invention is generally directed to the field of nail manicuring. More specifically, this invention applies to nail care products and processes for treating nails.

### DESCRIPTION OF RELATED TECHNOLOGY

Acrylic nails are used to artificially enhance the appearance of natural fingernails. Various forms of this product are used throughout the world, including press-on nails, two-part (liquid and powder) system, and UV gel system.

Press-on acrylic nails, introduced to the manicure industry in the early 1970s, are nail-shaped pieces of polymer that are glued onto natural nails. Subsequent developments afforded more natural-looking nail enhancements which bond to the real nail by an acrylic-based resin. Such a resin is created by mixing a liquid and powder together to form a thick paste. A nail technician applies the paste over the natural nail and allows it to harden to form a durable nail coating finish that is filed into the desired shape.

Although press-on acrylic nails are used today, they have been largely replaced by sculpted acrylic nails. Sculpted acrylic nails may be formed by several different methods, but the most popular and widely-used method comprises an application of a two-part acrylic formulation by a nail technician. The nail technician typically wets a brush with a monomer liquid, dips the wetted brush into a polymeric powder to form a wet ball of dough, places the wetted ball of the blend of monomer liquid and polymer powder onto a nail and an acrylic nail form adjacent to a nail, and shapes the artificial nail.

The acrylic nail form is a substrate used for the construction of acrylic nails beyond the existing free edge of the nail. Elongation requires affixing a substrate means to the exposed edge of the nail, which means has the general contour of the natural nail and extends from that place to the desired length and along the plane of the natural nail. The substrate is typically made of material to which the polymerized mixture does not adhere, so that the form may be removed following construction of the artificial nail.

The powder portion of the two-part acrylic formulation generally comprises a solid polymer, such as poly (methyl methacrylate), poly (ethyl methacrylate), copolymers of poly (methyl methacrylate) and poly (ethyl methacrylate), and a catalyst, such as benzoyl peroxide. The residual benzoyl peroxide contributes to the instability of the two-part system once it is mixed. The polymers are typically not crosslinked, which causes them to dissolve in the monomer liquid.

The two-part acrylic formulation also comprises a liquid, generally referred to as “monomer liquid,” which comprises an (meth)acrylic monomer, such methyl methacrylate or ethyl methacrylate, crosslinkers, additives, catalysts such as amine catalysts, and optionally other ingredients needed for forming a mixture with the solid polymer to prepare an artificial nail.

One of the biggest disadvantages of the monomer liquid used in the traditional system is the offensive odor of the ethyl methacrylate. This odor may be unappetizing to most clients, and its long-term exposure has been hypothesized to be dangerous to the nail technicians.

Odorless systems comprising hydroxyethyl methacrylate, hydroxypropyl methacrylate, and other methacrylates have been developed, but they generally have the disadvantage of poor cure and finished aesthetics.

Another method of forming gel nails is by the use of a UV-curable system. In one prior art method of the UV-curable system method, a brush dipped into the formulated monomer liquid is placed into the formulated polymer powder, applied to the client's fingernail, and after the artificial nails are formed, the artificial nails are cured under a UV lamp for several minutes.

A UV-cured system has no odor compared to traditional acrylic systems. However, many nail technicians find that UV-curable systems are difficult to work with and are presently typically only used to create overlays and not as freestanding nail extensions. Further, another disadvantage of typical UV-cured two-part powder systems is brittleness of the formed nail.

Gel systems, in contrast to the traditional polish and other polymer-type systems, particularly ultraviolet-cured gel systems, often comprise a gel that may be brushed onto the nails, cured, and shaped to create lifelike artificial nails. As compared with traditional polishes or other non-gel polymer-type systems, gel systems are relatively easy to use, are applicable in less time, are lightweight on the nail, have no odor (or only minimal odor), are durable, and have a high-quality shine.

A method of preparation of radiation-curable artificial nail gels is taught in U.S. Pat. No. 8,367,045. The method disclosed comprises preparing colored UV-curable artificial nail gel compositions comprising dispersing a pigment in an organic liquid to form a pigment concentrate and mixing the pigment concentrate with a polyfunctional acrylic monomer and/or a polyfunctional acrylic oligomer, resulting in highly colored artificial nail gels.

Photocurable nail compositions containing a dispersion of acrylic polymer particles is disclosed in U.S. Patent Application Publication No. 2017/0172883, 2017/0172890, and International Patent Publication No. WO 2017/112334. The photocurable nail composition comprises at least one dispersion of acrylic polymer particles and at least one inorganic gelling agent. Those publications also disclose a nail composition set comprising (1) at least one base coat composition; (2) at least one photocurable color coat composition comprising at least one dispersion of acrylic polymer particles and at least one inorganic gelling agent; and (3) at least one photocurable topcoat composition comprising at least one dispersion of acrylic polymer particles and at least one inorganic gelling agent.

A composition having a reduced exotherm in the actinic curing of urethane (meth)acrylate oligomers on fingernails are disclosed in U.S. Pat. No. 9,044,405. A nail coating composition having a reduced exotherm during actinic curing of the coating on a nail may be formed by the use of urethane(meth)acrylate. The composition may include a curable resin, a monomer, a photoinitiator, a chemical filter capable of absorbing ultraviolet (UV) light and reducing exotherm, and an additive.

Compositions for gel applications for nails comprising methacrylates, urethanes, and esters is disclosed in U.S. Pat. No. 9,023,326. A radiation curable gel nail coating composition according to that patent comprises about 50 wt % di-[hydroxyethyl methacrylic] trimethylhexyl dicarbamate, about 3 wt % methacrylic acid ester, about 3 wt % hydroxyethyl methacrylate, about 3 wt % hydroxypropyl methacrylate, and about 0.2 wt % of a photoinitiator.

Compositions and methods for UV-curable cosmetic nail coatings is disclosed in U.S. Pat. No. 8,901,199. Compositions for natural and artificial nail coatings that provide improved adhesion-promoting and improved solvent-susceptibility may comprise a polymerizable (meth)acrylate, a urethane (meth)acrylate resin and an adhesion promoter selected such as hydroxypropyl methacrylate, hydroxyethyl methacrylate, ethyl methacrylate, tetrahydrofurfuryl methacrylate, and like.

Nail enamel composition containing a urea-modified thixotropic agent in a solvent system are disclosed in U.S. Pat. No. 6,555,096. A nail enamel composition which contains, in a cosmetically acceptable solvent system containing diacetone alcohol and at least one additional solvent chosen from C₁-C₆ alkyl acetates and C₁-C₆ alkyl alcohols, at least one film-forming substance and at least one urea-modified thixotropic agent. The use of this thixotropic agent in the specific solvent system gives nail enamel compositions with higher gloss, high clarity, improved aesthetics in the bottle, excellent thixotropic properties, and improved application properties.

Nail compositions containing inorganic gelling agent and dispersion of acrylic polymer particles are disclosed in U.S. Patent Application Publication Nos. 2017/0172883, and 2017/0172890. A nail composition set comprises at least one base coat composition; at least one photocurable color coat composition comprising at least one dispersion of acrylic polymer particles and at least one inorganic gelling agent; and at least one photocurable topcoat composition comprising at least one dispersion of acrylic polymer particles and at least one inorganic gelling agent.

Although many advances in the art of formulating acrylic nail system have been made to solve various problems, overcoming problems associated with UV cured systems remain elusive.

### SUMMARY OF THE INVENTION

The present invention relates to a photopolymerizable composition for forming a cosmetic coating for nails comprising a continuous phase and a discontinuous phase. The continuous phase comprises monomers, crosslinkers, photoinitiators, and optionally oligomers and other additives. The discontinuous phase comprises particles such as polymers, inorganics or a mixture thereof.

More specifically, the present invention relates to a photopolymerizable composition for forming a cosmetic coating for nails comprising: about 20 wt % to about 80 wt % of a discontinuous phase mixture comprised of one or more classes of particles with the mean particle size between about 1 micrometer and 100 micrometers, wherein each class of particles is a polymer or an inorganic material; 0 wt % to about 80 wt % of one or more (meth)acrylate oligomers; about 5 wt % to about 60 wt % of one or more (meth)acrylate monomers; one or more crosslinkers; and one or more photoinitiators; wherein all wt % are with respect to the photopolymerizable composition.

The discontinuous phase polymer is selected from the group consisting of poly(C₁₋₁₂alkyl(meth)acrylate), a styrene, an aramid, a cellulose ester, an epoxy, a melamine-formaldehyde, a phenol-formadehyde, a poly(arylene sulfide), a poly(arylene terephthalamide), a polyacrylamide, a polyacrylonitrile, a polyalkylene, a polyamide, a polycarbonate, a polyester, a polyetheretherketone, a polyether, a polyether sulfone, a polyimide, a polylactam, a polylactone, a polyol, a polyphosphazene, a polysiloxane, a polyurea, a polyurethane, a polyvinyl alcohol, a polyvinyl halide, a polyvinyl acetate, a silicone, a wax, a rubber, a copolymer of any of the preceding,

The discontinuous phase inorganic material is selected from the group consisting of barium sulfate, boron nitrides, calcium sulfate, a ceramic, clay, diamond, glass, metal oxide, metal, mica, mineral, silicate, silicon dioxide, talc, titanium dioxide, an encapsulated version of any of the preceding, and a mixture thereof.

This composition has good storage stability and exhibits high viscosity. The composition of the present invention can be shaped readily by a nail technician to form nail coatings without any additional use of monomers liquid, followed by exposure to UV light to photopolymerize the composition to create a hard, durable nail coating.

The continuous phase portion of the composition of the present invention comprises at least a monomer, a crosslinker, a photoinitiator, and optionally an oligomer. Each or any of these four ingredients may be a single compound or each, or any of these ingredients may be a mixture of compounds that fall within the definition of such ingredient.

The particle size under one embodiment is within the range of 1 to 100 μm. Under another embodiment, the particle size is between about 5 to 50 micrometers. Under yet another embodiment the particle size is between about 5 to 30 micrometers. Such particles demonstrate superior performance over sub-micron particles or particles that are larger than about 100 μm.

Additional ingredients such as a colorant, a special effects pigment, or an antioxidant may be included in the composition of the present invention. These ingredients may be in either the continuous phase or discontinuous phase.

One of the advantages that the present invention provides is a high viscosity composition which is easy to apply. Another advantage of the composition of the present invention and the method of its using over the traditional acrylic nail formulation is that the composition is a one part formulation. Another advantage of the one-part formulation is the lack of heterogeneity in the formulations which may be associated with two-part formulations. A further advantage of the composition of the present invention is a lack of odor of the one-part formulation. Yet a further advantage of the present invention is the lower exotherm profile compared to the gel formulations. A still further advantage of the present invention is the excellent adhesion, abrasion resistance, and mechanical strength of the cured nail coating. Another advantage of the present invention is that unlike gel nail coverings which tend to shrink while curing, the composition of the present invention displays no significant shrinkage. A further advantage of the composition of the present invention is the stability of the one-part formulation. Yet another advantage of one embodiment of the present invention is the reduction of the yellowing of the cosmetic nail coating prepared from the composition of the present invention. Yet still another advantage of the present invention compared to the traditional two-part system is the long open time to sculpt the nail.

The photopolymerizable composition of the present invention composition consists of finely divided particles of the discontinuous phase distributed through the continuous phase. The continuous phase is a mixture of at least three ingredients, namely, (meth)acrylate monomers, crosslinkers, photoinitiators, and optionally (meth)acrylate oligomers. The discontinuous phase under an embodiment of the present invention comprises one or more classes of particles.

The particles of the discontinuous phase are selected so that the particles are compatible with any or all of the ingredients of the continuous phase. The compatibility of the particles with the continuous phase is characterized by inertness and insolubility.

The discontinuous phase mixture under one embodiment comprises one or more polymers. Such polymers may be crosslinked, or they may be uncrosslinked, as long as the polymers in the discontinuous phase are appreciably insoluble in the continuous phase.

The particle size of the components of the discontinuous phase mixture is in the range of several microns or tens of microns. Particles in micron range appear to yield better formulations than similar compositions wherein the particle size are less than about 1 micron which tend to act as a single continuous phase viscous fluid. Particles in the micron range also tended to perform better than the particles wherein the range is greater than about 100 microns, due to the faster settlement, the grittiness of the resulting nail coating, and other processing concerns.

When mixed with the other ingredients of the photopolymerizable composition of the present invention, the polymer typically does not dissolve, but forms a homogenous suspension of the polymer in the composition.

The powder before mixing with other ingredients may consist of fine particles. The particle is not limited by any shape. For example, the particle can be in the shape of a sphere, an oblate spheroid, a prolate spheroid, an irregular shape, a sheet, nanotubes, a fiber, a crystal, or a rod.

The (meth)acrylate monomer used in the present invention may be any acrylate monomer or methacrylate monomer that is used in nail art formulations in which the curing is performed by UV light. Examples of (meth)acrylate monomers include hydroxypropyl (meth)acrylate, hydroxyethyl (meth)acrylate, ethyl (meth)acrylate, propyl (meth)acrylate, butyl (meth)acrylate, isobutyl (meth)acrylate, sec-butyl (meth)acrylate, tetrahydrofurfuryl (meth)acrylate, caprolactone (meth)acrylate, (meth)acroyloxyethyl maleate, 2-hydroxyethyl (meth)acrylate/succinate, phthalic acid monoethyl (meth)acrylate, and isobornyl (meth)acrylate.

The continuous phase of the photopolymerizable composition of the present invention for forming a cosmetic coating for nails also optionally comprises one or more (meth)acrylate oligomers. Examples of (meth)acrylate oligomers include urethane (meth)acrylate, epoxy (meth)acrylate, epoxy urethane (meth)acrylate, (meth)acrylated acrylate, (meth)acrylated polyether, (meth)acrylated polycarbonate, (meth)acrylated cellulose, (meth)acrylated butadiene, (meth)acrylated styrene, polyester (meth)acrylate, polyester urethane (meth)acrylate, polyether urethane (meth)acrylate, polybutadiene urethane (meth)acrylate, and a mixture thereof.

The photopolymerizable composition of the present invention for forming a cosmetic coating for nails also comprises one or more crosslinkers. Examples of crosslinkers include diacrylates, triacrylates, tetraacrylates, pentaacrylates and higher acrylates.

In addition to the above-described (meth)acrylate-based polymerizable materials, other polymerizable monomers, oligomers or polymers of monomers which contain at least one free radical polymerizable group in the molecule may be used without any limitations in the curable gel.

The photopolymerizable composition of the present invention for forming a cosmetic coating for nails also optionally comprises one or more photoinitiators. As described below, the photoinitiator is selected so that it is activated by photons of the wavelength associated with UV light of the UV lamp. Preferably, the photoinitiator should be active at the wavelength of UV light of UV lamps commonly found in nail salons. Such photoinitiators may be selected from benzyl ketones, monomeric hydroxyl ketones, polymeric hydroxyl ketones, α-amino ketones, acyl phosphine oxides, metallocenes, benzophenone, and benzophenone derivatives.

The photopolymerizable composition of the present invention under one embodiment further comprises a small amount of a colorant or special effects pigment or a combination thereof.

Examples of pigments may be incorporated into the photopolymerizable composition of the present invention include ultramarine, manganese violet, zinc oxide, FD&C Blue No. 1, D&C Blue No. 4, Iron Blue, D&C Violet No. 2, and a mixture thereof. Special effects pigment may be any pigment that gives either the photopolymerizable composition or the formed cured composition a special effect, such as an increased pearlescent, iridescent, shimmering, transparency or complex effects.

The photopolymerizable composition of the present invention for forming a cosmetic coating for nails is a viscous liquid. It has a similar consistency as a mixture of polymer powder and monomer liquid in a traditional two-part system that is in the middle of the open time. Under one embodiment, the viscosity of the composition of the present invention is above 400,000 centipoise. Under another embodiment, the viscosity of the composition of the present invention is between about 500,000 to about 5,000,000 centipoise.

The present invention is also directed to the photopolymerizable composition wherein the composition exhibits a lower exotherm. Under one embodiment of the present invention, the exotherm (i.e., the peak temperature achieved during the reaction less the starting temperature) is less than about 30° C. (54° F.).

The photopolymerizable composition of the present invention under one embodiment is stable at 20° C. for at least 4 hours. Under another embodiment, the composition of the present invention is stable at 15° C. for at least 4 hours. Under yet another embodiment the composition of the present invention is stable in a dark container at 49° C. for at least four months. Under one embodiment the composition of the present invention is stable at 65° C. for at least two months.

The photopolymerizable composition of the present invention may be prepared by any means used to add and blend high viscosity compositions.

The container in which the photopolymerizable composition of the present invention is sold may be any container capable of handling high viscosity compositions and of providing protection from UV light. Examples of such a container include a wide-mouth jar, a tube container, a syringe container and a foil packet.

Another aspect of the present invention is a method of use of the above-described photopolymerizable composition to form a cosmetic coating for a nail. There are three methods which may be used to form the cosmetic nail coating.

The first method of forming the cosmetic nail coating comprises the steps of placing the above-described composition onto a nail of the client; and exposing the composition to UV light.

The second method of forming an acrylic nail comprises the steps of contacting the photopolymerizable composition with a liquid to create a blend; placing the blend onto a nail; and exposing the blend to UV light.

The third method of forming an acrylic nail comprises the steps of placing the photopolymerizable composition onto a nail; contacting the composition with a liquid; and exposing the mixture to UV light.

### DETAILED DESCRIPTION OF THE INVENTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation. Further, although certain methods are described with reference to certain steps that are presented herein in a certain order, in many instances, these steps may be performed in any order as may be appreciated by one skilled in the art, and the methods are not limited to the particular arrangement of steps disclosed herein.

As used herein and in the appended claims, the singular forms “a”, “an”, and “the” include plural references unless the context clearly dictates otherwise. The singular form of any class of the ingredients refers not only to one chemical species within that class, but also to a mixture of those chemical species; for example, the term “ photoinitiator” in the singular form, may refer to a mixture of compounds each of which is also a photoinitiator. The terms “a” (or “an”), “one or more” and “at least one” can be used interchangeably herein. It is also to be noted that the terms “comprising”, “including”, and “having” can be used interchangeably.

The term “or” is interpreted to mean “and/or”, unless the context indicates otherwise. Thus, a phrase “A or B” is interpret to cover embodiments having element A alone, element B alone, or elements A and B taken together.

The abbreviations and symbols as used herein, unless indicated otherwise, take their ordinary meaning. The abbreviation or symbol “cp” means centipoises or millipascal-seconds. The abbreviation or symbol “μm” means micrometer.

Any member in a list of species (such as compounds or polymers) that are used to exemplify or define a genus (such as a composition), may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genius just as well as, or better than, any other species listed.

The term “wt %” means percent by weight. The phrase “wherein all wt % are with respect to the photopolymerizable composition” means that the recited percent by weight values for each ingredient is compared to the entire photopolymerizable composition.

The term “about” when referring to a number means ±3%. For example, the phrase “about 40 wt %” refers to a number between and including 38.800 wt % and 41.200 wt %. The term “about” refers only to those value ranges that are physically or theoretically possible; for example, the phrase “about 99 wt %” refers to a number between and including 96.030 wt % and 100.000 wt %.

The modifier term “typical” in the phrase “typical storage conditions” of a composition, refers to storage conditions within the range experienced by at least one standard deviation (68.1%) of commercial units of the compositions under commercial conditions.

The term “client” refers to a person whose nails are being treated.

The phrase “nail technician” or “technician” is a worker skilled or licensed in the art of providing nail extensions, artificial nails, acrylic nails, gel nails, and other manicure services for clients. Alternative names for a nail technician may include a manicurist or a cosmetologist. Such a person may work for pay at a nail salon or may be a manicure aficionado.

Under one embodiment of the present invention, the client and the nail technician are two different individuals. Although the description of the invention below describes the nail technician and the client as two separate individuals, it is understood that the claimed invention and methods are also suitable for use by a single person who is both a nail technician and a client. Under another embodiment of the present invention, the client and the nail technician are the same person.

The terms “nail”, refer to either a fingernail or a toenail. The term “nail” also refers to a human nail, as well as to any toughened keratin at the end of a digit of a non-human animal. The phrase “cosmetic nail coating” refers to the hardened, fully cured substance covering a part or all of the nail, and any portions of this substance that extends or is built beyond the free edge of the nail.

The term “acrylic” in the phrase “acrylic nail” refers to hardened polymerized composition used in manicure arts, which are composed of any of several types of poly ((meth)acrylates), or copolymers of various (meth)acrylate monomers, oligomers or copolymers of various (meth)acrylate monomers with any of several non-(meth)acrylic monomers.

When referring to a composition, the definition of the term “acrylate” as referred to in the monomeric form, includes an ester, a salt, or a conjugate base of an acrylic acid, with the formula CH₂═CH—COO⁻. The definition of the term “acrylate” referred to in the polymeric or oligomeric form includes the repeating unit of an ester, a salt, or a conjugate base of an acrylic acid, with the formula —[CH₂—CH(COO)⁻]—.

The definition of the term “methacrylate” as referred to in the monomeric form includes an ester, a salt, or a conjugate base of methacrylic acid, with the formula CH₂═C(CH₃)—COO⁻. The definition of the term “methacrylate” as referred to in the polymeric or oligomeric form includes an ester, a salt, or a conjugate base of a methacrylic acid, with the formula —[CH₂═C(CH₃)—COO⁻]—.

The term “(meth)acrylate” means acrylate, methacrylate, or a mixture thereof. When referring to a compound, “(meth)acrylate” means an ester, a salt, or a conjugate base of an acrylic acid, with the formula CH₂═C(R)—COO⁻, wherein R is H, Me, or a mixture thereof. The definition of the term “(meth)acrylate” as referred to in the polymeric or oligomeric form includes an ester, a salt, or a conjugate base of methacrylic acid, with the formula —[CH₂═C(R)—COO⁻]—, wherein R is H, Me, or a mixture thereof. By extension, a monomer, oligomer, or polymer name containing as a part of its term the string “(meth)acrylate” should be interpreted as referring to the same monomer, oligomer, or polymer, that is an acrylate, methacrylate, or a mixture thereof. For example, the term “poly(C₁₋₁₂alkyl (meth)acrylate)” means “any of poly(C₁₋₁₂alkyl acrylate), poly(C₁₋₁₂alkyl methacrylate), and a mixture of poly(C₁₋₁₂alkyl acrylate) and poly(C₁₋₁₂alkyl methacrylate)”.

The term “mixture” as in the phrases “continuous phase mixture” or “discontinuous phase mixture” refers to a composition comprising one or several ingredients, unless the number of ingredients is clear from the context.

When referring to a composition as a compound or a polymer, the composition may be of any purity suitable for the purpose.

The present invention relates to a photopolymerizable composition for forming a cosmetic coating for nails comprising a continuous phase and a discontinuous phase. The continuous phase comprises monomers, oligomers, crosslinkers, photoinitiators, and optionally, other additives. The discontinuous phase comprises particles such as polymers, inorganics or a mixture thereof.

More specifically, the present invention relates to a photopolymerizable composition for forming a cosmetic coating for nails comprising: about 20 wt % to about 80 wt % of a discontinuous phase mixture comprised of one or more classes of particles with the mean particle size between about 1 micrometer and 100 micrometers, wherein the each class of particles is selected from the group consisting of a poly(C₁₋₁₂alkyl(meth)acrylate), a styrene, an aramid, a cellulose ester, an epoxy, a melamine-formaldehyde, a phenol-formadehyde, a poly(arylene sulfide), a poly(arylene terephthalamide), a polyacrylamide, a polyacrylonitrile, a polyalkylene, a polyamide, a polycarbonate, a polyester, a polyetheretherketone, a polyether, a polyether sulfone, a polyimide, a polylactam, a polylactone, a polyol, a polyphosphazene, a polysiloxane, a polyurea, a polyurethane, a polyvinyl alcohol, a polyvinyl halide, a polyvinyl acetate, a silicone, a wax, a rubber, a copolymer of any of the preceding, barium sulfate, boron nitrides, calcium sulfate, a ceramic, clay, diamond, glass, metal oxide, metal, mica, mineral, silicate, silicon dioxide, talc, titanium dioxide, an encapsulated version of any of the preceding, and a mixture thereof; 0 wt % to about 80 wt % of one or more (meth)acrylate oligomers; about 5 wt % to about 60 wt % of one or more (meth)acrylate monomers; one or more crosslinkers; and one or more photoinitiators; wherein all wt % are with respect to the photopolymerizable composition.

This composition has good storage stability and exhibits high viscosity that resembles the viscosity of a typical traditional two-part system roughly 4 minutes after a monomer liquid and a polymer have been mixed. The composition of the present invention can be shaped readily by a nail technician to form nail coatings without any additional use of monomers liquid, followed by exposure to UV light to photopolymerize the composition to create a hard, durable nail coating.

The continuous phase portion of the composition of the present invention comprises at least a monomer, a crosslinker, a photoinitiator, and optionally, a (meth)acrylate oligomer. Each or any of these four ingredients may be a single compound or each or any of these ingredients may be a mixture of compounds that fall within the definition of such ingredient.

The discontinuous phase portion of the composition of the present invention comprises at least one class of particles or powders. Such particles may be of one class, or type or species, of a polymer or of an inorganic material. The discontinuous phase may also be a mixture of one or more of the different classes, type, or species of a polymer or inorganic material.

The particle size under one embodiment is within the range of 1 to 100 μm. Under another embodiment, the particle size is between about 5 to 50 micrometers. Under yet another embodiment the particle size is between about 5 to 30 micrometers.

Additional ingredients such as a colorant, a special effects pigment, or an antioxidant may be included in the composition of the present invention. These ingredients may be in either the continuous phase or discontinuous phase.

The composition of the present invention solves one or more problems associated the formation of nail coating or nail extension using either procedure relying on gel nail formulations or acrylic nail formulations.

One of the advantages that the present invention provides is a high viscosity composition which is easy to apply. Compared to the low viscosity of nail gel formulations that may run outside of the sculpting area, the composition of the present invention has a high viscosity out of the container. The composition of the present invention does not move easily until it is pushed into a desired shape by a manicurist tool, such as a brush or a pusher. The pushing of the composition of the present invention into a desired shape may be done neat, or it may be done with the aid of liquid which lowers the viscosity of the composition. One advantage is that either in the neat form or as a mixture of a liquid, the composition of the present invention remains firm and does not run. The technician may optionally control the viscosity through the use of the suitable liquid applied until the composition of the present invention is cured by ultraviolet light. Thus, total control over the viscosity of the nail covering or nail extension may be attained.

Another advantage of the composition of the present invention and the method of its using over the traditional acrylic nail formulation is that the composition is a one part formulation. There is no need to mix the composition of the present invention with another part. By not spending time mixing the formulation or waiting for it to set to a workable viscosity, time serving the client is reduced, resulting in faster service. Further, the technician does not have to worry about getting the exact mixing ratios correct in order to get a workable composition.

Another advantage of the one-part formulation is the lack of heterogeneity in the formulations which may be associated with two-part formulations. The composition of the present invention is homogeneous, allowing for uniform applicability of the composition.

A further advantage of the composition of the present invention is a lack of odor of the one-part formulation. Traditional two-part acrylic systems typically exhibit very strong odors; for example, a common commercially available monomer composition that comprises a mixture of ethyl methacrylate, glycol HEMA-methacrylate, HEMA, benzophenone-1, and dimethyltolylamine, exhibits a severely strong odor. The composition of the present invention has little or no odor. This lack of odor is especially desirable in small nail salons and spas, where multiple nail technicians may work side by side for many hours in a tight environment, or in environments with limited air circulation.

Yet a further advantage of the present invention is the lower exotherm profile compared to the gel formulations. UV light initiated gel formulas create heat when curing. Such heat can be unpleasant or even painful to the client, especially if the product is applied in a thick layer, which results in an increase of the heat flux from the gel into the nail bed. It has been observed that the composition of the present invention exhibits a significantly lower exotherm of the polymerization reaction. Because of the lack of such a heat spike, the technician is able to apply the composition to the present invention in thicker layers when creating nail extensions.

A still further advantage of the present invention is the excellent adhesion, abrasion resistance, and mechanical strength of the cured nail coating.

Another advantage of the present invention is that unlike gel nail coverings which tend to shrink while curing, the composition of the present invention displays no significant shrinkage.

A further advantage of the composition of the present invention is the stability of the one-part formulation. Because the composition is thermally stable and needs UV light to cure it, the composition of the present invention under one embodiment is shelf stable for several days, under another embodiment is shelf stable for several months, and under still another embodiment is shelf stable for several years.

Yet another advantage of one embodiment of the present invention is the reduction of the yellowing of the cosmetic nail coating prepared from the composition of the present invention. Unlike many other compositions that are thermally cured, or UV cured, the composition of the present invention, when cured, does not appreciably yellow.

Yet still another advantage of the present invention compared to the traditional two-part system is the long open time to sculpt the nail. The composition may be worked by the technician for as long as is necessary before the composition is exposed to UV light.

The above advantages listed above are illustrative and may not be exhaustive. Further, not every embodiment of the present invention necessarily displays all of the advantages listed.

Although there are various ways of describing multiphase systems comprising a wide variety of ingredients of various solubilities and miscibilities with respect to each other, for the sake of clarity, the description of the photopolymerizable composition of the present invention is simplified to a two-phase system.

The photopolymerizable composition of the present invention composition consists of finely divided particles, droplets, or bubbles of the discontinuous phase distributed through the continuous phase. The continuous phase is a mixture of at least three ingredients, namely, (meth)acrylate monomers, crosslinkers, photoinitiators, and optionally, (meth)acrylate oligomers. The discontinuous phase under an embodiment of the present invention comprises one or more classes of particles.

The weight ratio of the continuous phase to the discontinuous phase is in a range of about 10:90 to about 67:33. Under one embodiment the weight ratio is in the range of about 20:80 to about 60:40. Under another embodiment, the weight ratio is in the range of about 30:70 to about 50:50.

Because any ingredient (whether recited or not) is soluble, partially soluble, or is insoluble in the continuous phase, then the wt % of the continuous phase and wt % of the discontinuous phase necessarily add to 100 wt %.

Ingredients that are not soluble in the continuous phase, yet are not considered particles (such as gases, or insoluble liquids), are considered to be a part of the discontinuous phase. The discontinuous phase may thus comprise not only the claimed particles, but additional discontinuous material. Likewise, the continuous phase may comprise not only the (meth)acrylate monomers, crosslinker, photoinitiators, and optionally (meth)acrylate oligomers, but also additional continuous material.

The particles of the discontinuous phase are selected so that the particles are compatible with any or all of the ingredients of the continuous phase. The compatibility of the particles with continuous phase is characterized by inertness and insolubility. Such particles are in the range of 1 to 100 μm, or within the range of 5 to 50 μm, or within the range of 5 to 30 mu.

Firstly, the composition of the discontinuous phase is appreciably inert with respect to the continuous phase, or any ingredients therein under the typical storage conditions. The phrase “appreciably inert” means that there is little or no chemical reaction between the discontinuous phase and the continuous phase when the composition of the present invention under typical storage conditions. However, during the use of the formulation of the present invention, especially when exposed to the actinic light source, it is acceptable and appropriate for the particles of the discontinuous phase to react with any ingredients in the continuous phase. Under one embodiment, no more than about 10 wt % of the discontinuous phase reacts with the continuous phase or any parts thereof under typical storage conditions. Under another embodiment, no more than about 3 wt % of the discontinuous phase reacts with the continuous phase or any parts thereof under typical storage conditions. Under a still further embodiment, no more than about 1 wt % of the discontinuous phase reacts with the continuous phase or any parts thereof under typical storage conditions.

Secondly, the composition of the discontinuous phase is appreciably insoluble with respect to the continuous phase. The phrase “appreciably insoluble” means that there is little or no dissolving of the discontinuous phase in the continuous phase when the composition of the present invention under typical storage conditions. Under one embodiment, no more than about 10 wt % of the discontinuous phase dissolves in the continuous phase under typical storage conditions. Under another embodiment, no more than about 3 wt % of the discontinuous phase dissolves in the continuous phase under typical storage conditions. Under a still further embodiment, no more than about 1 wt % of the discontinuous phase dissolves in the continuous phase under typical storage conditions.

The discontinuous phase mixture under one embodiment comprises one or more polymers. Such polymers may be crosslinked, or they may be uncrosslinked, as long as the polymers in the discontinuous phase are appreciably insoluble in the continuous phase.

The term “particles” is taken at its broadest meaning. The particles may act as fine pieces of solid in the continuous phase, or they may act is fine fluid dispersion.

The particle size of the components of the discontinuous phase mixture are in the range of several microns or tens of microns. Particles in micron range appear to yield better formulations than similar compositions wherein the particle size are less than about 1 micron which tent to act as a single continuous phase viscous fluid. Particles in the micron range also tended to perform better than the particles wherein the range is greater than about 100 microns, due to the faster settlement, the grittiness of the resulting nail coating, and other processing concerns.

Under one embodiment the mean particle size is within the range of about 1 μm and about 100 μm. Under another embodiment, the mean particle size is within the range of about 5 μm and about 50 μm. Under another embodiment, the mean particle size is within the range of about 5 μm and about 30 ρm.

The particle size may be determined by any appropriate method, such as ASTM E2651. The phrase “particle size” is the mean particle size, such as mean diameter over volume, D[4,3], or de Broukere mean. The mean particle size may be determined by routine testing, such as on the Horiba Laser Scattering Particle Size Distribution Analyzer LA-950.

When mixed with the other ingredients of the photopolymerizable composition of the present invention, the polymer typically does not dissolve, but forms a homogenous suspension of the polymer in the composition.

Under one embodiment of the present invention, the particles are composed of a polymer. The particles may be composed of any polymer that does not react appreciably under storage condition with any of the ingredients of the continuous phase. Examples of such polymers include a poly(C₁₋₁₂alkyl(meth)acrylate), a styrene, an aramid, an aramid, a cellulose ester, an epoxy, a melamine-formaldehyde, a phenol-formaldehyde, a poly(arylene sulfide), a poly(arylene terephthalamide), a polyacrylamide, a polyacrylonitrile, a polyalkylene, a polyamide, a polycarbonate, a polyester, a polyetheretherketone, a polyethers, a polyether sulfone, a polyimide, a polylactam, a polylactone, a polyol, a polyphosphazene, a polysiloxane, a polyurea, a polyurethane, a polyvinyl alcohol, a polyvinyl halide, a polyvinyl acetate, a silicone, a wax, a rubber, a copolymer of any of the preceding, an encapsulated version of any of the preceding, and a mixture thereof.

For readability purposes, the polymers listed above are in their adjective form; for each of the adjective, a polymer or a resin is assumed. For example, the phrase “an aramid” is meant to represent the phrase “an aramid polymer”, “an aramid resin”, or other similar phrases as are known to practitioners in the solid polymer art.

For each of the polymers listed above and throughout, the definition of the polymer is not limited to any single species, but is rather meant to represent the genus which may be classified as such polymer.

Further, each of the polymer genera listed above and throughout is meant to represent homopolymers and copolymers.

A polymer specie may fall within more than one genera. For example, a polymer that contains both ether bonds and ester bonds may fall within the definition of both a polyether and a polyester.

An aramid is an aromatic polyamide. An example of an aramid is a polymer with a formula —[NH—C₆H₄—NH—CO—C₆H₄—CO]—. Aramids may be formed by a condensation polymerization of terephthaloyl chloride with p-phenylenediamine. Examples of aramids include para-aramids (sold under marks such as Kevlar, Technora, Twaron, Heracron) and meta-aramids (such as Nomex, Teijinconex). Commercially available aramid powders include TWARON 5011 (a poly(paraphenylene terephthalamide) polymer powder, 55 micrometer average particle size, 97% solids); TWARON 1088 (a poly(paraphenylene terephthalamide) 250 micrometer chopped fiber, 100% solids); both available from Teijin Twaron USA Inc., Conyers, Ga., USA.

A cellulose ester is a polymer that is frequently used in the nail care products. Under one embodiment, the composition of the present invention comprises cellulose ester that is in a form of a powder which is suspended in the continuous phase. Examples of a cellulose ester include cellulose acetate alkylate, cellulose acetate (CA), cellulose propionate (CP), cellulose butyrate (CB), cellulose acetate propionate (CAP), and cellulose acetate butyrate (CAB). Examples of the utility of such cellulose esters can be found in Prog. Polym. Sci. 2001, 26, 1605-1688. Cellulose esters are generally prepared by first converting cellulose to a cellulose triester before hydrolyzing the cellulose triester in an acidic aqueous media to the desired degree of substitution (the number of substituents per anhydroglucose monomer). Aqueous acid-catalyzed hydrolysis of cellulose triacetate yields a random copolymer that can consist of 8 different monomers depending upon the final degree of substitution (see, for example, Macromolecules 1991, 24, 3050).

Under one embodiment, the term “cellulose ester” also includes the derivatives of cellulose esters. Examples of cellulose ester derivatives include carboxymethyl cellulose esters such as those described for example in U.S. Pat. Nos. 5,668,273; 5,792,856; and 5,994,530. These cellulose derivatives are cellulose ether esters in which an intervening ether linkage attaches a carboxylate to the anhydroglucose units of the cellulose chain. These derivatives are formed by esterifying carboxymethyl cellulose (an ether) to the fully substituted carboxymethyl cellulose ester followed by hydrolysis to the desired ester degree of substitution. This type of carboxylated cellulose esters offers the advantage of a non-hydrolysable carboxylate linkage.

Another type of carboxylated cellulose esters is those in which the carboxylate functionality is attached to the cellulose backbone via an ester linkage. An example of this class is cellulose acetate phthalate and the like which are described in U.S. Pat. No. 3,489,743. In general, these cellulose ester derivatives are formed by first preparing a neutral, randomly substituted cellulose ester, e.g., a CA, with the desired degree of substitution. In a second reaction, the carboxylate functionality is installed by treating the cellulose ester with an anhydride such as phthalic anhydride.

An additional type of carboxylated cellulose esters is those, which result from ozonolysis of cellulose esters in the solid state (Sand, I. D., Polymer Material Science Engineering 1987, 57-63; U.S. Pat. No. 4,590,265). Ozonolysis of cellulose ester provides a polymer that contains not only carboxylates but also aldehydes, ketone, and peroxides as well. The process results in significant loss in polymer molecular weight and relatively low levels of oxidation. Furthermore, the process is not specific in that any of the cellulose ester hydroxyls can be oxidized.

An epoxy is a three-dimensional cross-linked thermoset obtained by curing an uncured epoxy resin. The epoxy is obtained by curing an uncured epoxy resin with itself, or by forming a copolymer with polyfunctional curatives or hardeners.

Uncured epoxy resins are formed from monomers or prepolymers comprising epoxide groups. Examples of uncured epoxy resins include bisphenol A epoxy (produced from combining epichlorohydrin and bisphenol A to give bisphenol A diglycidyl ethers), bisphenol F epoxy (produced from combining epichlorohydrin and bisphenol F to give bisphenol F diglycidyl ethers), glycidylamine epoxy (produced by reacting aromatic amines with epichlorohydrin), aliphatic epoxy (produced by glycosylation of aliphatic alcohols or polyols), and novolac epoxy (produced by a reaction of phenols with formaldehyde and subsequent glycosylation with epichlorohydrin to yield epoxidised novolacs).

Examples of hardeners include amines (including aliphatic amines, cycloaliphatic amines, and aromatic amines), anhydrides (such as cyclic anhydrides), phenols (such as bisphenols or novolacs), and thiols.

A melamine-formaldehyde resin (MF resin) is a thermosetting polymer class of compounds exemplified by hexa-hydroxymethyl derivatives prepared by a condensation of formaldehyde with melamine. Examples of MF resins include methylated melamine formaldehyde resins and methylol melamine formaldehyde resins. Powdered MF resins may be commercially available from various manufacturers, such as Hexion, Arclin, Georgia-Pacific, Ineos, BASF, and DIC.

A phenol-formaldehyde resin (PF resin) is any of several synthetic polymers obtained by the reaction of phenol or substituted phenol with formaldehyde.

A poly(arylene sulfide) is any of several polymers consisting of aromatic rings linked with sulfides, such as —[C₆R₄—S]ₙ—. Examples of poly(arylene sulfides) include aromatic polythiols, and polyphenyl thioethers. Powdered poly(arylene sulfides) may be available under tradenames such as Ryton®, Tedur®, LNP™ KONDUIT, Torelina™ and Fortron®.

A poly(arylene terephthalamide) is any of several polymers synthesized in solution from the monomers arylene diamine and terephthaloyl chloride in a condensation reaction. Example of arylene diamine includes phenylenediamine, such as p-phenylenediamine and m-phenylenediamine. Examples of poly(arylene terephthalamide) include polymers comprising —[NH—C₆H₄—NH—CO—C₆H₄—CO]—.

A polyacrylamide is any of several high molecular weight polymers formed from acrylamide or its derivatives. Examples of polyacrylamides include poly(2-propenamide), polymers with structure —[CH₂—CH(—C(O)—NH₂)]ₙ—, and salts thereof. The polyacrylamide may have a linear chain structure or a cross-linked structure. Powdered polyacrylamide may be commercially available from various manufacturers, such as BASF, Capital Resin, Kemira, Shangdong Tongli, and SNF Floerger.

A polyacrylonitrile is any of several synthetic, polymer resin, with formula —[CH2—CH(CN)]ₙ—. Examples of polyacrylonitriles include copolymers such as poly(styrene-acrylonitrile) and poly(acrylonitrile butadiene styrene) polymers.

A polyalkylene is any of several polymers produced from alkylenes. A polyalkylene is the same or similar to polyolefin, and have the formula —[CH₂—CHR]ₙ—. Examples of polyalkylene include polyethylene, PE, polypropylene, PP, polymethylpentene, PMP, polybutene, PB-1, polyisobutylene, PIB, ethylene propylene rubber, EPR, ethylene propylene diene monomer rubber, EPDM rubber, and various copolymers thereof. Further examples include: poly(butylene), poly(butyl ethylene), poly(cyclohexylethylene), poly(ethylene), poly(heptylethylene), poly(hexylethylene), poly(isobutene), poly(isobutylethylene), poly(isopropylethylene), poly(2-methylbutene), poly(octylethylene), poly(pentylethylene), poly(propylene), poly(propylethylene), and poly(tert-butylethylene).

A polyamide is any of several polymers wherein the repeating units are linked by amide bonds. A polyamide may have repeating units such as —[NH—CO—R]ₙ—, —[NH—R₁—NH—CO—R₂—CO]—, and like. Polyamides can be made through step-growth polymerization or solid-phase synthesis. Examples of polyamines include polylactam, Nylon 3, poly(propiolactam), Nylon 6, Poly(caprolactam), Nylon 10, poly(decano-10-lactam), Nylon 11, poly(undecano-11-lactam), Nylon 12, poly(dodecano-12-lactam), Nylon 6,6, poly(hexamethylene adipamide), Nylon 6,10, poly(hexamethylene sebacamide), Nylon 6,12, poly(hexamethylene dodecanediamide), Nylon 8, polycapryllactam, poly(hexane-1,6-diyl teraphthalamide), polyaramide, poly(m-phenylene terephthalamide), and copolymers thereof.

A polycarbonate is any of several thermoplastic polymers containing carbonate groups in its chemical structures. Polycarbonates (PC) have repeating units such as —[O—R—O—CO]ₙ—. Polycarbonates may contain the precursor monomer bisphenol A (BPA). Examples of polycarbonates include poly(bisphenol A carbonate), poly(4,4′-thiodiphenylene carbonate), poly(bisphenol B carbonate), poly(bisphenol F carbonate), poly(ethylene carbonate), poly(propylene carbonate), poly(2,6,3′,5′-tetrachloro bisphenol A carbonate), poly(tetramethyl bisphenol A carbonate), and copolymers thereof.

A polyester is any of several thermoplastic polymers containing ester groups in the main chain. The term polyester includes both a thermoplastic polyester and a thermoset polyester. The polyester may be prepared by azeotrope esterification, alcoholic transesterification, acylation, acetate esterification, or ring-opening polymerization.

Under one embodiment, the polyester is aliphatic, such as homopolymeric (e.g., polyglycolide or polyglycolic acid, PGA, polylactic acid, PLA, polycaprolactone, PCL, polyhydroxyalkanoate, PHA, polyhydroxybutyrate, PHB) or copolymeric (polyethylene adipate, PEA, polybutylene succinate, PBS, poly(3-hydroxybutyrate-co-3-hydroxyvalerate), and PHBV).

Under another embodiment, the polyester is a semi-aromatic or an aromatic copolymer, such as polyethylene terephthalate, PET, polybutylene terephthalate, PBT, polytrimethylene terephthalate, PTT, polyethylene naphthalate, PEN, and Vectran.

Examples of polyesters include poly(bisphenol A isophthalate), poly(bisphenol A terephthalate), poly(butylene adipate), poly(butylene isophthalate), poly(butylene sebacate), poly(butylene succinate), poly(butylene terephthalate), poly(ethylene sebacate), poly(ethylene succinate), poly(caprolactone), poly(cyclohexylenedimethylene terephthalate), poly(ethylene adipate), poly(ethylene isophthalate), poly(ethylene naphthalate), poly(ethylene phthalate), poly(ethylene terephthalate), polyglycolide, poly(hexylene sebacate), poly(3-hydroxybutyrate), poly(4-hydroxybutyrate), polylactic acid, poly(trimethylene succinate), poly(trimethylene terephthalate)poly(bisphenol A isophthalate), poly(bisphenol A terephthalate), poly(butylene adipate), poly(butylene isophthalate), poly(butylene sebacate), and copolymers thereof.

A polyetheretherketone, or polyether ether ketone, or PEEK, is a polyaryletherketone thermoplastic polymer. Polyetheretherketone polymers may be obtained by step-growth polymerization of the dialkylation of bisphenolate salts.

A polyether is any of several thermoplastic polymers containing ether groups in the main chain. Examples of polyether polymers include paraformaldehyde polyoxymethylene, POM, polyacetal, polyformaldehyde, polyethylene oxide, PEO, polyoxyethylene, POE, polypropylene oxide, PPOX, polyoxypropylene, POP, polytetramethylene ether glycol, PTMEG, and other polyethers with high molar mass. A polyether repeating units include —CH₂—, —[CH₂—CH₂—O]—, —[CH₂—C(CH₃)—O]—, —[CH₂—CH₂—CH₂—CH₂—O]—, and like. Examples of polyethers include polyglycol, polyacetal-polyoxymethylene, poly(3-butoxypropylene oxide), poly(epichlorohydrin), poly(ethylene glycol), poly(hexamethylene oxide), poly(3-methoxypropylene oxide), poly[oxy(hexyloxymethyl)ethylene], poly(oxymethylene-oxyethylene), poly(oxymethylene-oxytetramethylene), polypropylene glycol), poly(tetrahydrofuran), poly(trimethylene glycol), and poly[1,1-bis(chloromethyl)trimethylene oxide].

A polyether sulfone, or a polyethersulfone, or a polysulfone, is any of several of thermoplastic polymers that contain the subunit -[aryl-SO₂-aryl]-. A polysulfone is produced by the reaction of a diphenol and bis(4-chlorophenyl)sulfone, forming a polyether by the elimination of sodium chloride. Examples of a polyethersulfone include poly(ether ether sulfone), poly(ethersulfone), poly(phenylsulfone), and bisphenol A polysulfone. Polysulfones may be commercially available from Solvay Specialty Polymers, BASF, and PolyOne Corporation.

A polyimide is any of several polymers of imide monomers, with general structure —[R—CO—NR′—CO]—. The polyimide may be aliphatic, semi-aromatic, or aromatic. A polyimide may be either thermoplastic or thermoset polymer. A polyimide may be formed by the reaction between a dianhydride and a diamine, or by the reaction between a dianhydride and a diisocyanate.

A polylactam is a polymer made from lactams or cyclic amides.

A polylactone is any of several polymers comprising the repeating unit —[(CH₂)ₘ—CO—O]—. Polylactones may be prepared by ring opening polymerization of lactone using a catalyst such as stannous octoate. Various polylactones may be obtained commercially under the mark InstaMorph, Polymorph, Shapelock, ReMoldables, Plastdude or TechTack.

A polyol is any of several polymers that comprise multiple hydroxyl groups.

A polyphosphazene is any of several polymers that include a range of hybrid inorganic-organic polymers comprising alternating phosphorus and nitrogen atoms. Such polymers comprise the repeating unit —[N═PX₂]—, wherein X is stabilizing ligand, such as —OR, —NHR, —OAr, and like. The polyphosphazene may be linear a polymer, a cyclolinear polymer, a cyclomatrix polymer, etc.

A polysiloxane, or a silicone, is any of several polymers that include any polymer with siloxane repeating units, —[O—SiR₂]—, wherein R is an organic group, such as an alkyl group. Polysiloxanes may be prepared by hydrolysis of dialkyldichlorosilane. Examples of polysiloxane include poly(diethylsiloxane), PDES, poly(dimethylsiloxane), PDMS, poly(methylphenylsiloxane, PMPS, and copolymers thereof.

A polyurea is any of several polymers that include any elastomers that are produced through step-growth polymerization from the reaction product of an isocyanate and a synthetic resin blend component. The isocyanate may be aromatic or aliphatic. The isocyanate can be a monomer, an oligomer, a polymer, quasi-prepolymer, or a prepolymer. The prepolymer, or quasi-prepolymer, can be made of an amine-terminated polymer resin, or a hydroxyl-terminated polymer resin. The resin blend may be made up of amine-terminated polymer resins, and/or amine-terminated chain extenders.

A polyurethane is any of several polymers that include any polymers comprising urethane linkages, or carbamate linkages, or linkages with a general formula —NH—CO—O—. Such polyurethane polymers may be thermosetting polyurethanes or thermoplastic polyurethanes. Polyurethane polymers may be formed by reacting a polyisocyanate with a polyol. The polyisocyanates (or simply isocyanates) and polyols used to make polyurethanes contain, on average, two or more functional groups per molecule.

A polyvinyl alcohol is any of several polymers that include polymers comprising units —[CH₂—CHOH]— and derivatives thereof. Such polymers may be used as an emulsion polymerization aid to make polyvinyl acetate dispersions. Polyvinyl alcohol also includes in its meaning derivatives or reaction products thereof, such as polyvinyl acetals, which are prepared by reacting aldehydes with polyvinyl alcohol. Examples include poly(4-vinyl phenol), poly(4-hydroxystyrene), polyvinyl butyral, PVB, polyvinyl formal, PVF, and Formvar. Such derivatives may be prepared from polyvinyl alcohol by reaction with butyraldehyde, formaldehyde, and like.

A polyvinyl halide is any of several polymers that include polymers comprising repeating units such as —[CH₂—CHX]—, or generally, —[C(H,X)—C(H,X)]—, wherein X is a halide. Examples of polyvinyl halide are polyvinyl chloride, polyvinyl, and PVC. Polyvinyl chloride may be rigid or flexible. Polyvinyl may be produced by polymerization of the vinyl chloride monomer. Polyvinyl halide may be produced by suspension polymerization, emulsion polymerization, or bulk polymerization. Suspension polymerization of PVC yields particles with average diameters of 100 to 180 μm. Emulsion polymerization of PVC yields a particle size of around 0.2 μm. Examples of polyhaloolefins include poly(chlorotrifluoroethylene), poly(tetrafluoroethylene), poly(vinyl bromide), poly(vinyl chloride), poly(vinyl fluoride), poly(vinylidene chloride), poly(vinylidene fluoride), and like.

A polyvinyl acetate is any of several aliphatic rubbery synthetic polymer that includes units —[CH₂—CH(O—(C═O)—CH₃)]—. Polyvinyl acetate is an example of a polyvinyl ester polymer which comprises units with the general formula —[(C═O)R—O—CH—CH₂]—. Polyvinyl acetate may be a thermoplastic. Polyvinyl acetate may be prepared by the polymerization of vinyl acetate monomer, i.e., free radical vinyl polymerization of the monomer vinyl acetate.

The term “wax” refers to a ternary or a quaternary wax-resin composite that is suitable for use in cosmetic preparations. Under one embodiment, a wax-resin composite may be made by melting thermoplastic hydrocarbon resin, optionally with an antioxidant. The melted thermoplastic hydrocarbon resin and waxes may be blended at a temperature sufficient to melt the waxes. After blending and melting, the blended thermoplastic hydrocarbon resin and waxes are allowed to solidify. Solidification is followed by post-processing the wax-resin composite mixture to form slabs, pastilles, flakes or other forms. A method for producing a wax-resin composite comprises at least partially solvating a thermoplastic resin in a solvent to form a resin-solvent blend. This procedure may be done at a heat of 80 to 85° C. A composition of molten wax is blended with the resin-solvent blend. The resin-solvent blend and a molten wax are blended to form a wax-resin blend. This is followed by removing the solvent from said wax-resin blend.

The term “rubber” refers to polymers based on the latex, or polymers or cis-1,2-polyisoprene. The rubber as used as particles of the discontinuous phase include rubbers that are elastomers, thermoplastic, or thermoset. The definition includes both unvulcanized and vulcanized rubber, as long as the rubber particles are unreactive at storage with the ingredients of the continuous phase. The term also refers to rubber compositions that comprise fillers, such as factice, whiting, carbon black, and similar ingredients.

A poly(meth)acrylate is any of several polymers that comprise units of formula —[CH₂═C(R)—COOR′]—, wherein R is H, Me, or a mixture thereof, and R′ is an organic group. Examples of poly(meth)acrylates include polymers such as poly(C₁₋₁₂alkyl(meth)acrylate) polymer, poly(C₁₋₁₂alkyl(meth)acrylate) copolymer, styrene polymer, styrene copolymer, and mixtures thereof. The composition of the present invention comprises about 20 wt % to about 80 wt % of a polymer selected from the group consisting of poly(C₁₋₁₂alkylacrylate) polymer, poly(C₁₋₁₂alkyl methacrylate) polymer, poly(C₁₋₁₂alkylacrylate) copolymer, poly(C₁₋₁₂alkyl methacrylate) copolymer, a styrene polymer, a styrene copolymer, and mixtures thereof.

The phrase “poly(C₁₋₁₂alkyl(meth)acrylate) polymer” means a polymer which is comprised mostly or exclusively of C₁₋₁₂alkyl(meth)acrylate monomer residues, or polymer units. Under one embodiment most or all of the C₁₋₁₂alkyl(meth)acrylate polymer units are the same. Under another embodiment, the C₁₋₁₂alkyl(meth)acrylate polymer units are different from each other. An example of “poly(C₁₋₁₂alkyl(meth)acrylate) polymer” includes a polymer that comprises methyl acrylate units, methacrylate units, and methyl methacrylate units.

The phrase “poly(C₁₋₁₂alkyl(meth)acrylate) copolymer” means a copolymer of C₁₋₁₂alkyl(meth)acrylate polymer units with other polymer units. The C₁₋₁₂alkyl(meth)acrylate polymer units may be the same, or they may vary. The phrase “other polymer units” means polymer units that do not fall within the definition of poly(C₁₋₁₂alkyl(meth)acrylate); examples of such, include styrene, divinyl benzene, and a mixture of styrene and divinyl benzene.

Under one embodiment, the C₁₋₁₂alkyl(meth)acrylate polymer units in the poly(C₁₋₁₂alkyl(meth)acrylate) copolymer are the same units. Under another embodiment, the C₁₋₁₂alkyl(meth)acrylate polymer units in the poly(C₁₋₁₂alkyl(meth)acrylate) copolymer are different polymer units. An example of “poly(C₁₋₁₂alkyl(meth)acrylate) copolymer” includes a polymer that comprises methyl acrylate units, methyl methacrylate units, and styrene units.

The phrase “styrene polymer” means a polymer consisting essentially of —[CH₂—CHPh]— polymer units. The phrase “polymer unit” is synonymous with a “monomer residue”, or with a “repeating unit”.

The phrase “styrene copolymer” means a copolymer of styrene units with other polymer units that are not styrene. Examples of “styrene copolymer” includes styrene/(meth)acrylate copolymer, and styrene/(meth)acrylate/divinylbenzene copolymer.

A copolymer comprising divinylbenzene is generally used in amounts as needed for crosslinking. Under one embodiment, the solid polymer comprises little or no crosslinking. Under another embpdment, the solid polymer comprises a significant amount of crosslinking, which may mitigate the solubility of the polymer.

Examples of poly(C₁₋₁₂alkyl (meth)acrylate) include poly(methyl (meth)acrylate), poly(ethyl (meth)acrylate), poly(propyl (meth)acrylate), poly(n-propyl (meth)acrylate), poly(isopropyl (meth)acrylate), poly(butyl (meth)acrylate), poly(n-butyl (meth)acrylate), poly(isobutyl (meth)acrylate), poly(sec-butyl (meth)acrylate), poly(pentyl (meth)acrylate), poly(hexyl (meth)acrylate), poly(heptyl (meth)acrylate), poly(octyl (meth)acrylate), poly(nonyl (meth)acrylate), poly(decyl (meth)acrylate), poly(undecyl (meth)acrylate), and poly(dodecyl (meth)acrylate). In these examples, the alkyl groups hexyl, heptyl, octyl, nonyl, decyl, undecyl, and dodecyl may be straight chain groups or branched groups.

Under one embodiment the poly(C₁₋₁₂alkyl (meth)acrylate) polymer or copolymer thereof is a copolymer of poly(C₁₋₁₂alkyl (meth)acrylate). Examples of such copolymers include styrene/C₁₋₁₂alkyl (meth)acrylic copolymer, styrene/C₁₋₁₂alkyl (meth)acrylate, styrene/C₁₋₁₂alkyl (meth)acrylate/divinylbenzene copolymers, and styrene/PEG-10 maleate/nonoxynol-10 maleate/acrylate copolymer.

Examples of poly(C₁₋₄alkyl (meth)acrylate) include poly(methyl (meth)acrylate), poly(ethyl (meth)acrylate), poly(propyl (meth)acrylate), poly(n-propyl (meth)acrylate), poly(isopropyl (meth)acrylate), poly(butyl (meth)acrylate), poly(n-butyl (meth)acrylate), poly(isobutyl (meth)acrylate), and poly(sec-butyl (meth)acrylate).

Under one embodiment of the present invention, the polymer consists of a single type of polymer. Under an alternative embodiment, the polymer consists of a mixture of poly(C₁₋₁₂alkyl acrylate) polymers; a mixture of poly(C₁₋₁₂alkyl methacrylate) polymers; a mixture of poly(C₁₋₁₂alkyl acrylate) polymer and poly(C₁₋₁₂alkyl methacrylate) polymer; a mixture of poly(C₁₋₁₂alkyl acrylate) polymers and poly(C₁₋₁₂alkyl methacrylate) polymer; a mixture of poly(C₁₋₁₂alkyl acrylate) polymers and poly(C₁₋₁₂alkyl methacrylate) polymer; a mixture of poly(C₁₋₁₂alkyl acrylate) polymers and poly(C₁₋₁₂alkyl methacrylate) polymers.

Examples of a suitable polymer comprise poly(methyl methacrylate), poly(ethyl methacrylate), and a mixture thereof. A suitable polymer is poly(methyl methacrylate), or PMMA.

Under one embodiment, PMMA is blended with other polymers to improve its flexibility.

The polymer is a powder which may be prepared by a routine technique, such as suspension polymerization in which the reaction takes place between droplets of the corresponding monomer suspended in a solution of water and catalyst.

The polymer suitable for use in the present invention may be any molecular weight, including a molecular weight that is similar to the molecular weight of polymers used in the acrylic nail industry.

Under one embodiment of the present invention, the particles are composed of an inorganic material. The particles may be composed of any inorganic material that does not react appreciably under storage condition with any of the ingredients of the continuous phase. Examples of such inorganic materials include barium sulfate, boron nitride, calcium sulfate, a ceramic, clay, diamond, glass, metal oxide, metal, mica, mineral, silicate, silicon dioxide, talc, titanium dioxide, an encapsulated version of any of the preceding, and a mixture thereof.

Barium sulfate is an inorganic compound comprised of BaSO₄. In the powder state, barium sulfate is an odorless, water-insoluble white crystalline solid. It occurs as the mineral barite, which is the main commercial source of barium and materials prepared from it.

Boron nitride is a compound of boron and nitrogen with the chemical formula BN. Under one embodiment, boron nitride exists in a crystalline form that is isoelectronic to a similarly structured carbon lattice. The particles of boron nitride may have any shape, including a hexagonal form corresponding to graphite, and is therefore used as a lubricant and an additive to cosmetic products. The particles of boron nitride may have a cubic shape or a sphalerite structure.

Calcium sulfate is an inorganic compound with the formula CaSO₄. Calcium sulfate also refers to any of the hydrates thereof. Examples of calcium sulfate include CaSO₄, calcium sulfate anhydrite, CaSO₄.2H₂O, calcium sulfate dihydrate, CaSO₄.0.5H₂O, calcium sulfate hemihydrate; and CaSO₄.xH₂O.

A ceramic is an inorganic, non-metallic, solid material comprising metal, non-metal or metalloid atoms primarily held in ionic and covalent bonds. Ceramic may be either crystalline, semi-crystalline, or non-crystalline. Examples of ceramics include oxides, titanates, borides, oxynitrides, carbides, nitrides, silicates, and like.

A clay is a finely-grained natural rock or soil material, composed of phyllosilicate minerals containing variable amounts of water trapped in the mineral structure. Examples of clays include kaolinite, montmorillonite-smectite, illite, and chlorite. Kaolinite is a layered silicate mineral with structure Al₂Si₂O₅(OH)₄. Montmorillonite is a subclass of smectite, a 2:1 phyllosilicate mineral characterized as having greater than 50% octahedral charge. Particles of montmorillonite are plate-shaped with an average diameter around 1 μm and a thickness of 9.6 nm. Illite is a secondary mineral precipitate phyllosilicate or layered alumino-silicate, wherein the structure is a 2:1 clay of silica tetrahedron—alumina octahedron—silica tetrahedron layers, with the chemical formula of [K,H₃O](Al,Mg,Fe)₂(Si,Al)₄O₁₀[(OH)₂,(H₂O)]. Chlorites are a group of phyllosilicate minerals, such as clinochlore, (Mg₅Al)(AlSi₃)O₁₀(OH)₈, chamosite, (Fe₅Al)(AlSi₃)O₁₀(OH)₈, nimite, (Ni₅Al)(AlSi₃)O₁₀(OH)₈, pennantite, and (Mn,Al)₆(Si,Al)₄O₁₀(OH)₈.

Diamond is a crystalline carbon, where the carbon atoms are arranged in a variation of the face-centered cubic crystal structure. Diamond powder can be from a natural or a synthetic source. Diamond powder is commercially available in a variety of sizes, including submicron, 1.2 to 2.3 μm, 3.1 to 6.2 μm, 3.9 to 7.8 μm, 4.7 to 9.3 μm, 6.2 to 9.3 μm, 7.8 to 15.5 μm, 9.3 to 17.1 μm, 15.5 to 23.3 μm, 17.1 to 27.9 μm, 27.9 to 41.9 μm, mesh sizes 15000#, 8000#, 5000#, 4000#, 3000#, 2000#, 1500#, 1200#, 800#, 700#, 600#, 400#, and like.

A glass is a non-crystalline amorphous solid comprised mostly of silicon dioxide, SiO₂, with minor amounts of several oxides and carbonates, such as sodium oxide, Na₂O, sodium carbonate, Na₂CO₃, calcium oxide, lime, CaO, and like. Glass is often transparent and has widespread practical, technological, and decorative usage in, for example, window panes, tableware, and optoelectronics. The most familiar, and historically the oldest, types of glass are “silicate glasses” based on the chemical compound silica (silicon dioxide, or quartz)

A metal is a metal element or an alloy of metal elements. Examples of common metal powders include aluminum powder, iron powder, nickel powder, steel powders, brass powder, copper powder, bronze powder, nickel silver powder, zinc powder, silver powder, titanium powder, and like. Additional metals include about 80 transition metal and main group metals, and alloys thereof. Metal powders come in a wide variety of particle size.

A metal oxide is any of the compositions of one or more oxygen atoms combined with at least one transition metal, a main group metal or both.

Mica is a phyllosilicate mineral composition with formula X₂Y₄₋₆Z₈O₂₀(OH, F)₄, in which X is K, Na, Ca, Ba, Rb, or Cs; Y is Al, Mg, Fe, Mn, Cr, Ti, Li, etc.; and Z is Si or Al. The mica could be a dioctahedral mica (such as muscovite), or a trioctahedral mica (such as biotite, lepidolite, phlogopite, zinnwaldite, and clintonite. The powder particles of mica are in the form of flakes.

A mineral is any of 5,000 naturally occurring chemical compounds, usually of crystalline form and abiogenic in origin

A silicate is a compound containing an anionic silicon group, such as [SiO₄]⁴⁻. Such silicates constitute the majority of the Earth's crust. Examples of silicates include both mineral and synthetic materials. Examples of silicates include nesosilicates (e.g., olivine), sorosilicates, —[Si₂O₇]⁶⁻ (e.g., epidotes and melilites), cyclosilicates, —[SiₙO₃ₙ]²ⁿ⁻ (e.g. tourmalines), inosilicates, —[SiₙO₃ₙ]²ⁿ⁻ (e.g., pyroxenes), inosilicates, —[Si₄ₙO₁₁ₙ]⁶ⁿ⁻ (e.g. amphiboles), phyllosilicates, —[Si₂ₙO₅ₙ]²ⁿ⁻ (e.g., micas and clays), tectosilicates —[AlₓSi_{y}O_{2x+2y}]^{x-} (e.g., quartz, feldspars, and zeolites).

Silicon dioxide is an oxide of silicon with the chemical formula SiO₂. Examples of silicates include both naturally occurring and synthetic materials. Examples of silicates (i.e., silicon dioxides) include fused quartz, fumed silica, silica gel, and aerogels.

Talc is a clay mineral composed of hydrated magnesium silicate. The chemical formula for talc includes H₂Mg₃(SiO₃)₄, and Mg₃Si₄O₁₀(OH)₂.

Titanium dioxide is a naturally occurring oxide of titanium with the chemical formula TiO₂.

The powder before mixing with other ingredients may consist of fine particles. The particle is not limited by any shape. For example, the particle can be in the shape of a sphere, an oblate spheroid, a prolate spheroid, an irregular shape, a sheet, nanotubes, a fiber, a crystal, or a rod.

When mixed with the monomers, crosslinkers, and other ingredients, these particle are insoluble in the composition. The particles are said to be insoluble when less than 5% of the diameter of the microsphere is lost to the solution. Generally, insoluble particles gain weight and size as they swell upon exposure to the monomers and other ingredients.

The polymer particles may also further comprise other components such as radical initiators.

The photopolymerizable composition of the present invention is a two-phase composition. The continuous phase is a mixture of at least three ingredients, namely, (meth)acrylate monomers, crosslinkers, photoinitiators, and, optionally, (meth)acrylate oligomers.

The continuous phase of the photopolymerizable composition of the present invention for forming a cosmetic coating for nails comprises one or more (meth)acrylate monomers.

The (meth)acrylate monomer used in the present invention may be any acrylate monomer or methacrylate monomer that is used in nail art formulations in which the curing is performed by UV light. The (meth)acrylate monomer has a formula CH₂═C(R)—COOR′, wherein R is H, Me, or a mixture thereof, and R′ is an organic group. Examples of organic group R′ include hydrocarbons, that is, aliphatic (e.g., alkyl or alkenyl), alicyclic (e.g., cycloalkyl, cycloalkenyl) compounds, and aromatic-, aliphatic-, and alicyclic-substituted aromatic compounds, as well as cyclic compounds wherein the ring is completed through another portion of the molecule (e.g., two substituents together form an alicyclic compound); groups that include hetero atoms, that is, groups that contain other than carbon in a ring or chain otherwise composed of carbon atoms, such as oxygen, nitrogen, such as groups containing non-hydrocarbon groups, such as alkoxy, amino, amido, and similar groups.

Examples of (meth)acrylate include methyl acrylate, methyl methacrylate, ethyl acrylate, ethyl methacrylate, hydroxypropyl acrylate, hydroxypropyl methacrylate, butylacrylate, butyl methacrylate, hydroxyethyl acrylate, propyl methacrylate, isobutyl methacrylate, sec-butyl methacrylate, hydroxyethyl methacrylate, butoxyethyl acrylate, butoxyethyl methacrylate, diethylaminoethyl acrylate, diethylaminoethyl methacrylate, 2-ethylhexyl acrylate, 2-ethylhexyl methacrylate, ethoxyethyl acrylate, ethoxyethyl methacrylate, t-butyl aminoethyl acrylate, t-butyl aminoethyl methacrylate, methoxyethylene glycolacrylate, methoxyethylene glycol methacrylate, phosphoethyl acrylate, phosphoethyl methacrylate, methoxy propyl acrylate, methoxy propyl methacrylate, phenoxyethylene glycol acrylate, tetrahydrofurfuryl methacrylate, phenoxyethylene glycol methacrylate, caprolactone methacrylate, methacroyloxyethyl maleate, 2-hydroxyethyl methacrylate/succinate, phthalic acid monoethyl methacrylate, phenoxypolyethylene glycol acrylate, phenoxypolyethylene glycol methacrylate, 2-hydroxy-3-phenoxypropyl acrylate, 2-hydroxy-3-phenoxypropyl methacrylate, isobornyl acrylate, isobornyl methacrylate, 3-chloro-2-hydroxypropyl acrylate, 3-chloro-2-hydroxypropyl methacrylate, and mixtures thereof.

Under one embodiment, the at least one or more (meth)acrylate monomers comprise a (meth)acrylate selected from the group consisting of hydroxypropyl (meth)acrylate, hydroxyethyl (meth)acrylate, ethyl (meth)acrylate, propyl (meth)acrylate, butyl (meth)acrylate, isobutyl (meth)acrylate, sec-butyl (meth)acrylate, tetrahydrofurfuryl (meth)acrylate, caprolactone (meth)acrylate, (meth)acroyloxyethyl maleate, 2-hydroxyethyl (meth)acrylate/succinate, phthalic acid monoethyl (meth)acrylate, isobornyl (meth)acrylate, or a mixture thereof.

Under one embodiment of the present invention, the at least one of the one or more (meth)acrylate monomers comprises a hydroxyl-containing (meth)acrylate monomer.

The continuous phase of the photopolymerizable composition of the present invention for forming a cosmetic coating for nails comprises 0 wt % to about 80 wt % of one or more (meth)acrylate oligomers.

Under one embodiment the presence of (meth)acrylate oligomers is optional. In such a case, the amount of (meth)acrylate oligomers is 0 wt % or essentially 0 wt %.

Under one embodiment the composition of the present invention comprises less than 1 wt % of one or more (meth)acrylate oligomers.

Under another embodiment, the composition of the present invention comprises about 20 wt % to about 80 wt % of one or more (meth)acrylate oligomers.

Under one embodiment of the present invention, the (meth)acrylate oligomer consists of a single type of oligomer. Under an alternative embodiment, the (meth)acrylate oligomers consist of a mixture of oligomers.

The molecular weight of the (meth)acrylate oligomer suitable for use in the present invention is similar to the (meth)acrylate oligomers used in the acrylic nail industry.

The functionality of a (meth)acrylate oligomer is about 2 to about 30.

Examples of (meth)acrylate oligomers include urethane (meth)acrylate, epoxy (meth)acrylate, epoxy urethane (meth)acrylate, (meth)acrylated acrylate, (meth)acrylated polyether, (meth)acrylated polycarbonate, (meth)acrylated cellulose, (meth)acrylated butadiene, (meth)acrylated styrene, polyester (meth)acrylate, polyester urethane (meth)acrylate, polyether urethane (meth)acrylate, polybutadiene urethane (meth)acrylate, and a mixture thereof.

Urethane (meth)acrylate comprises at least two acryl or methacryl groups and at least one urethane group. Urethane (meth)acrylate may also comprise additional functional groups, such as an ester, an ether, and like. The phrase “urethane (meth)acrylate” thus also includes polyester urethane acrylate, polyester urethane methacrylate, polyether urethane acrylate, polyether urethane methacrylate, and like.

Examples of urethane (meth)acrylate oligomers include: aliphatic urethane (meth)acrylates, aromatic urethane (meth)acrylates, polyester urethane (meth)acrylates, and polyether polyols and aliphatic, aromatic, polyester, polyether diisocyanates capped with (meth)acrylate end-groups, epoxy (meth)acrylates, epoxy urethane (meth)acrylates.

Epoxy acrylates and epoxy methacrylates may be based on aliphatic or aromatic epoxy prepolymers capped with acrylate or methacrylate end-groups.

Acrylated polyester oligomers, useful in one embodiment of the present invention, have at least two or more acrylate or methacrylate groups and a polyester core. Acrylated polyether oligomers have at least two or more acryl or methacryl groups and a polyether core. Acrylated acrylate oligomers have at least two or more acryl or methacryl groups and a polyacrylic core.

Urethane (meth)acrylate can be can be prepared by any conventional means, such as one of two modes of addition. A polyol can be terminated on each end with a diisocyanate and then capped with a hydroxy functional (meth)acrylate. Alternatively, a diisocyanate can first be reacted with a hydroxy functional (meth)acrylate, and that product can be used to terminate a polyol.

Under one embodiment the oligomers is an aliphatic urethane acrylate. Under an alternative embodiment, the oligomers is a polyester urethane acrylate. Under still another embodiment the oligomers is a mixture of is a mixture of an aliphatic urethane acrylate oligomer and a polyester urethane acrylate oligomer.

The photopolymerizable composition of the present invention for forming a cosmetic coating for nails also comprises one or more crosslinkers. A crosslinker is a compound that contains two or more (meth)acrylate groups. Crosslinkers are necessary to provide chemical bonding between several monomers, oligomers, polymers or combinations thereof to yield a cross-linked polymeric structure needed for the formation of cosmetic nail coating.

Under one embodiment the monomer liquid comprises a single crosslinker. Under another embodiment, the monomer liquid comprises two or more different compounds that are crosslinkers.

Examples of crosslinkers include diacrylates, triacrylates, tetraacrylates, pentaacrylates and higher acrylates. Such examples include trimethylolpropane triacrylate, trimethylolethane triacrylate, trimethylolpropane trimethacrylate, trimethylolethane trimethacrylate, tetramethylene glycol dimethacrylate, triethylene glycol dimethacrylate, tetraethylene glycol diacrylate, pentaerythritol diacrylate, penta-erythritol triacrylate, pentaerythritol tetraacrylate, dipentaerythritol diacrylate, dipenta-erythritol triacrylate, dipentaerythritol tetraacrylate, dipentaerythritol pentaacrylate, dipenta-erythritol hexaacrylate, tripentaerythritol octaacrylate, pentaerythritol dimethacrylate, penta-erythritol trimethacrylate, dipentaerythritol dimethacrylate, dipentaerythritol tetramethacrylate, tripentaerythritol octamethacrylate, ethylene glycol diacrylate, 1,3-butanediol diacrylate, 1,3-butanediol dimethacrylate, sorbitol triacrylate, sorbitol tetraacrylate, pentaerythritol-modified triacrylate, sorbitol tetramethacrylate, sorbitol pentaacrylate, sorbitol hexaacrylate, oligoester acrylates and methacrylates, glycerol di- and tri-acrylate, 1,4-cyclohexane diacrylate, bisacrylates and bismethacrylates of polyethylene glycol, and mixtures thereof.

Under one embodiment, at least one of the one or more crosslinker is selected from the group consisting of trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, dipentaerythritol penta(meth)acrylate and ethoxylated iscyanuric acid tri(meth)acrylates.

Under one embodiment the crosslinkers comprise methacrylate groups. Example of such crosslinkers include dimethacrylates, trimethacrylates, tetramethacrylate, and higher methacrylates. Examples of such methacrylic crosslinkers include trimethylolpropane trimethacrylate, trimethylolethane trimethacrylate, tetramethylene glycol dimethacrylate, triethylene glycol dimethacrylate, pentaerythritol dimethacrylate, pentaerythritol trimethacrylate, dipentaerythritol dimethacrylate, dipentaerythritol tetramethacrylate, tripentaerythritol octamethacrylate, 1,3-butanediol dimethacrylate, sorbitol tetramethacrylate, oligoester methacrylates, bis methacrylate of polyethylene glycol having a molecular weight of from 200 to 1500, and mixtures thereof. For example, trimethylolpropane trimethacrylate is a composition consisting of, or comprising largely of, the compound of formula (CH₂═CMe—C(O)—O—CH₂)₃C—C₂H₅. It is a low volatility trifunctional monomer offering fast cure response in free radical polymerization.

Another suitable crosslinker is an alkoxylated crosslinker, with the formula (CH₂═CMe—C(O)—O—(AO)ₓ—CH₂—)₃C—R; wherein wherein R is a C₁ to C₆ alkyl group; AO is a small alkoxy group, such as an ethylene oxide, —CH₂—CH₂—O—, propylene oxide, —CH(CH₃)—CH₂—O—, —CH₂—CH₂—CH₂—O—, butylene oxide, and —CH(Et)—CH₂—O—; and wherein for each (CH₂═CMe—C(O)—O—(AO)ₓ—CH₂—) group x is independently 0, 1, 2, or 3. Using R=ethyl, and AO=ethylene oxide as an example, an exemplary alkoxylated crosslinker has a structure of formula

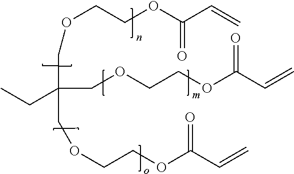

wherein m, n, and o are each independently 0, 1, 2, or 3.

Under one embodiment the composition of the present invention comprises (a) about 50 wt % to about 60 wt % of one or more poly(C₁₋₁₂alkyl(meth)acrylate) polymers; (b) about 25 wt % to about 35 wt % of one or more (meth)acrylate oligomers; (c) about 10 wt % to about 15 wt % of one or more (meth)acrylate monomers, and a crosslinker. This composition may optionally also include other ingredients.

Under one embodiment of the present invention, in this 50-60 wt % polymer: 25-35 wt % oligomer: 10-15 wt % monomer composition, the polymers are insoluble in the composition; the oligomer comprises a urethane (meth)acrylate oligomer; and the monomers comprises a monomer that is hydroxyalkyl (meth)acrylate, cycloalkyl (meth)acrylate, and a mixture of both.

Under one embodiment the crosslinker in this composition is a tri((meth)acrylate), and the composition further comprising a photoinitiator.

In addition to the above-described (meth)acrylate-based polymerizable materials, other polymerizable monomers, oligomers or polymers of monomers which contain at least one free radical polymerizable group in the molecule may be used without any limitations in the curable gel. Typical examples include esters of acrylic and methacrylic acid, herein termed (meth)acrylic ester. Specific but not limiting examples of mono (meth)acryloyl esters include methyl (meth)acrylate, ethyl (meth)acrylate, hydroxypropyl (meth)acrylate, butyl (meth)acrylates, hydroxy ethyl (meth)acrylates, butoxyethyl (meth)acrylate, diethylaminoethyl (meth)acrylate, 2-ethylhexyl (meth)acrylate, ethoxyethyl (meth)acrylate, t-butyl aminoethyl (meth)acrylate, methoxyethylene glycol (meth)acrylate, phosphoethyl (meth)acrylate, methoxy propyl (meth)acrylate, methoxy polyethylene glycol(meth)acrylate, phenoxyethylene glycol (meth)acrylate, phenoxypolyethylene glycol (meth)acrylate, 2-hydroxy-3-phenoxypropyl (meth)acrylate, 2-(meth)acryloxyethylsuccinic acid, 2-(meth)acryloylethylphthalic acid, 2-(meth)acryloyloxypropylphthalic acid, stearyl (meth)acrylate, isobornyl (meth)acrylate, 3-chloro-2-hydroxypropyl (meth)acrylates, tetrahydrofufuryl (meth)acrylate, methacryloyloxyethyl trimelilitc anhydride, (meth)acrylamides and allyl monomers. Specific but not limiting examples of difunctional (meth)acryloyl esters include 1,4 butane diol di(meth)acrylate, 1,6 hexananediol di(meth)acrylate, alkoxylated hexane diol di(meth)acrylate, 1,9 nonanediol di(meth)acrylate, 1,10 decanediol di(meth)acrylate, neopentyl glycol di(meth)acrylate, alkoxylated neopentyl glycol di(meth)acrylate, 2-methyl-1,8-octane diol di(meth)acrylate, cyclohaxane dimethanol di(meth)acrylate, glycerin di(meth)acrylate, ethylene glycol di(meth)acrylate, triethyleneglycol di(meth)acrylate, polyethylene glycol di(meth)acrylate, propylene glycol di(meth)acrylate, polypropylene glycol di(meth)acrylate, ethoxylated propylene glycol di(meth)acrylate, ethoxylated polypropylene glycol di(meth)acrylate, polyethoxypropoxy di(meth)acrylate, ethoxylated bisphenol A di(meth)acrylate, propoxylated bisphenol A di(meth)acrylate, propoxylated ethoxylated bisphenol A di(meth)acrylate, bisphenol A glycidyl methacrylate, tricyclodecanedimethanol di(meth)acrylate, glycerin di(meth)acrylate, ethoxylated glycerin di(meth)acrylate, bis acrylamides, bis allyl ethers and allyl (meth)acrylates. Examples of tri and or higher (meth)acryloyl esters include trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, dipentaerythritol penta(meth)acrylate and ethoxylated iscyanuric acid tri(meth)acrylates. Monomers containing acid groups may also be used including (meth)acrylic acid, bis(gyceryl dimethacrylate)pyromellitate, pyromellitic dimethacrylate, methacryloyloxyethyl phthalate, methacryloyloxyehtyl maleate, 2 hydroxyethyl methacrylate/succinate, 1,3 glyceryl dimethacylate maleate adduct, and 1,3 glyceryl dimethacrylate succinate adduct. Partially aminated monomers and oligomers may also be used. These are prepared by reaction of amines, preferably secondary amines, with some of the (meth)acryloyl groups of the multifunctional monomers or oligomers.

The photopolymerizable composition of the present invention for forming a cosmetic coating for nails also optionally comprises one or more photoinitiators. As described below, the photoinitiator is selected so that it is activated by photons of the wavelength associated with UV light of the UV lamp. Preferably, the photoinitiator should be active at the wavelength of UV light of UV lamps commonly found in nail salons.

Such photoinitiators may be selected from benzyl ketones, monomeric hydroxyl ketones, polymeric hydroxyl ketones, α-amino ketones, acyl phosphine oxides, metallocenes, benzophenone, and benzophenone derivatives. Specific examples of photoinitiators include 1-hydroxy-cyclohexylphenylketone; benzophenone; 2-benzyl-2-(dimethylamino)-1-(4-(4-morphorlinyl)phenyl)-1-butanone; 2,2-dimethoxy-2-phenyl acetophenone; 2-methyl-1-(4-methylthio)phenyl-2-(4-morphorlinyl)-1-propanone; 2,4,6-trimethylbenzoyldiphenyl-phosphine oxide; bis(2,4,6-trimethyl benzoyl)phenyl phosphine oxide; diphenyl-(2,4,6-trimethylbenzoyl)phosphine oxide; bis(2,6-dimethoxybenzoyl-2,4,4-trimethyl pentyl)phosphine oxide; 2-hydroxy-2-methyl-1-phenyl-propan-1-one; phenyl bis(2,4,6-trimethylbenzoyl)phosphine oxide; benzyl-dimethylketal; isopropylthioxanthone; bis(η⁵-2,4-cyclopentadien-1-yl)bis[2,6-difluoro-3-(1H-pyrrol-1-yl)phenyl]titanium), and mixtures of any of the foregoing.

Under one embodiment of the present invention, the photopolymerizable composition comprises a single chemical compound that exhibits photoinitiating properties. Under an alternative embodiment, the photoinitiator is a mixture of photoinitiators.

The photoinitiator is present in the photopolymerizable composition in amounts sufficient to be effective in aiding curing of the photopolymerizable composition. Such amounts may be determined empirically. The photopolymerizable composition comprises up to about 10 wt % of one or more photoinitiators. Under one embodiment, the photopolymerizable composition comprises about 0.5 to about 5.0 wt % of one or more photoinitiators.

The photopolymerizable composition of the present invention under one embodiment further comprises a small amount of a colorant or special effects pigment or a combination thereof.

One purpose of using pigment in the photopolymerizable composition is to provide a tint or a color to the formed cosmetic nail coating. The use of such color in the photopolymerizable composition may allow the technician to omit certain selected post-treatment steps after the formation of the cosmetic nail coating.

Another purpose of using a pigment is to give a clear or colorless or whitish appearance of the cosmetic nail coating. The pigment may be used to address any yellowing of the cosmetic nail coating.

Yet another purpose of using a pigment is to provide a whitish appearance to the photopolymerizable composition, so that it appears as an attractive, clean product to the nail technician.

Examples of pigments may be incorporated into the photopolymerizable composition of the present invention include: annatto, caramel, carmine, β-carotene, potassium sodium copper chlorophyllin (chlorophyllin copper-complex), dihydroxyacetone, bismuth oxychloride, guaiazulene, iron oxides, ferric ammonium ferrocyanide, ferric ferrocyanide, chromium hydroxide green, chromium oxide greens, guanine, pyrophyllite, mica, silver, titanium dioxide, aluminum powder, bronze powder, copper powder, ultramarines, manganese violet, zinc oxide, luminescent zinc sulfide, FD&C Blue No. 1, D&C Blue No. 4, Iron Blue, D&C Brown No. 1, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, FD&C Red No. 40, D&C Violet No. 2, Ext. D&C Violet No. 2, FD&C Yellow No. 5, FD&C Yellow No. 6, D&C Yellow No. 7, Ext. D&C Yellow No. 7, D&C Yellow No. 8, D&C Yellow No. 10, D&C Yellow No. 11, and mixture of any of the preceding. As will be recognized by the practitioner of the art, some of the pigments in the above list are better suited for use in the photopolymerizable composition than others, because they offer better composition stability of the photopolymerizable composition, and they do not interfere with the UV curing process.

Under one embodiment the photopolymerizable composition comprises the pigment is selected from the group consisting of ultramarine, manganese violet, zinc oxide, FD&C Blue No. 1, D&C Blue No. 4, Iron Blue, D&C Violet No. 2, and a mixture thereof.

Special effects pigment may be any pigment that gives either the photopolymerizable composition or the formed cured composition a special effect, such as an increased pearlescent, iridescent, shimmering, transparency or complex effects. Examples of special effect pigments include titanated micas, mica-based interference colors, mica coated with titanium dioxide and iron oxide, mica-based gold pearls, mica-based metallic pearls, mica-based pearl pigments, bismuth oxychloride, synthetic mica-based interference pearls, synthetic mica-based white pigment, silicate-based pearls, titanium oxide and tin oxide on silicate platelets, flaked aluminum powder, silver coated silicate flakes, and any combination of the foregoing.

The photopolymerizable composition of the present application may further comprise additional ingredients, including colorants, dyes, whiteners, perfumes, thixotropes, stabilizers, anti-oxidants, and like.

Any of above polymers, oligomers, monomers, crosslinkers, pigments, and other ingredients as provided by its manufacturer may contain small amounts of additives and impurities. The purity level of the ingredient may be above 90%. Alternatively, the purity level may be above 95%. Under some embodiments, the purity level may be above 97%. Additives for monomers may include inhibitors such as hydroquinone, HQ, monomethyl ether quinone, MEHQ, isoascorbic acid, IA, butylated hydroxytoluene, BHT, and BHT adducts. Impurities may include isomers of the monomer, oligomers, unreacted starting materials, water, and solvent used in the formation of the monomers.

The photopolymerizable composition of the present invention for forming a cosmetic coating for nails is a viscous liquid. It has a similar consistency as a mixture of polymer powder and monomer liquid in a traditional two-part system that is middle of the open time.

Under one embodiment of the present invention, the viscosity of the photopolymerizable composition is greater than about 400,000 centipoise as measured by a cone and plate rheometer. The viscosity is determined by a cone and plate rheometer using 40 mm 1° steel cone with the truncation gap of 31 microns operating at 2 to 10 s⁻¹ shear rate on a 500 mg sample. The temperature of the Peltier plate is set 25° C., and the sample is conditioned to 25° C. The cone and plate rheometer may be any suitable rheometer for measuring high viscosity fluids, such as TA Instruments AR1500EX Rheometer. The viscosity is determined from measurements at 5 points from 2 s⁻¹ to 10 s⁻¹ shear rates.

Under one embodiment, the viscosity of the composition of the present invention is above 400,000 centipoise. Under another embodiment, the viscosity of the composition of the present invention is between about 500,000 to about 5,000,000 centipoise.

The present invention is also directed to the photopolymerizable composition wherein the composition exhibits a lower exotherm. The exotherm is the apparent rise in temperature due to the heat generated during the curing reaction caused by UV light. The exotherm is measured on a 0.5-gram sample formed on a glass substrate into a shape approximating a sculpted nail. The exotherm is measured by a thermocouple on the glass substrate to measure the approximate rise in temperatures that a client would feel during the curing process under the UV light.

The exotherm may be decreased by increasing the wt % of the polymer portion of the formulation. Further, the exotherm may be decreased by decreasing the wt % of the photoinitiators.

Under one embodiment of the present invention, the exotherm (i.e., the peak temperature achieved during the reaction less the starting temperature) is less than about 30° C. (54° F.). Under another embodiment, the exotherm is less than about 20° C. (36° F.). Under still another embodiment the exotherm is less than about 15° C. (27° C.).

The photopolymerizable composition of the present invention under one embodiment is stable in a dark container at 49° C. for four months, or at 65° C. for 2 weeks, or is stable for 49° C. for four months and at 65° C. for 2 weeks. Under one embodiment the composition of the present invention is stable at 49° C. for at least four months. Under another embodiment, the composition of the present invention is stable at 65° C. for at least two weeks. Under another embodiment, the composition of the present invention is stable at 49° C. for at least four months and at 65° C. for at least two weeks.

Under another embodiment, the composition of the present invention is stable at 20° C. for at least 4 hours. Under yet another embodiment the composition of the present invention is stable at 15° C. for at least 4 hours.

The stability may be tested in an enclosed container that does not let in any UV light. The term “stable” refers to the lack of change in the physical, chemical or esthetic properties of the photopolymerizable composition that would make the photopolymerizable composition unsuitable for sale to the consumer or would unsuitable for use by the technician. Exemplary changes of physical, chemical or esthetic properties of the photopolymerizable composition include polymerization, noticeable yellowing, a noticeable rise in viscosity.

The photopolymerizable composition of the present invention may be prepared by any means used to add and blend high viscosity compositions. The components may be added together in any order that is convenient from the engineering viewpoint. Addition of the components, blending of the components and filling appropriate containers may be done at elevated temperatures. Such temperatures should be less than temperatures which would initiate curing of the components.

The container in which the photopolymerizable composition of the present invention is sold may be any container capable of handling high viscosity compositions and of providing protection from UV light. One example of such a container is a wide-mouth jar. Another example of a suitable container is a tube container. Another example of a suitable container is a syringe container. Still another example of a suitable container is a foil packet.

Another aspect of the present invention is a container comprising the previously described photopolymerizable composition, wherein the container is a tube container or a syringe container, wherein the container delivers a uniform bead of the composition via an orifice.

A tube container of the present application comprises the previously described photopolymerizable composition inside of a tube. The tube is a cylindrical, hollow piece with a round or oval profile, similar to tubes used to contain toothpaste, ointment, adhesives, caulk, and other viscous liquids. The tube has an orifice which is designed to deliver a bead of the photopolymerizable composition. Such an orifice may be a part of a nozzle, which may be optionally capped or closed.

A syringe container of the present application comprises the previously described photopolymerizable composition in a syringe. The syringe of the present invention comprises a plunger that fits tightly into a barrel. The plunger can be pushed along inside the barrel, allowing the syringe to expel the photopolymerizable composition through an orifice at the open end of the tube. The orifice is designed to deliver a bead of the photopolymerizable composition. Such an orifice may be a part of a nozzle, which may be optionally capped or closed.

Foil packet, such as MylarFoil MiniPouches™, Stick Pack, Stikpak™, and like, deliver a dosage for a complete, ten fingernail treatment. One of the advantages of using foil pack is the ease of clean up. Another advantage is the uniform delivery of the composition.

The orifice is designed so that the technician is able to deliver a uniform bead onto the nail of the client. The phrase “uniform bead” means that the weight of the bead formed does not vary by more than 10 wt % from another bead when forced through the orifice using the same conditions (the same pressure and same length of time) as subjectively applied by the technician.

Another aspect of the present invention is a method of use of the above-described photopolymerizable composition to form a cosmetic coating for a nail. There are three methods which may be used to form the cosmetic nail coating.

The first method of forming the cosmetic nail coating comprises the steps of placing the above-described composition onto a nail of the client; and exposing the composition to UV light.

The placement of the bead of the photopolymerizable composition may be performed by the technician directly by squeezing it from a tube container or pushing it from a syringe with a plunger. Alternatively, the bead of the photopolymerizable composition may be done with the help of an acrylic or gel brush, such as a No. 8, 10, or 12 brush.

After the placement of the bead of the photopolymerizable composition onto the nail, the technician works the composition to move it into a desired location and form it into a desired shape.

After the composition is shaped, the composition, along with finger and the hand of the client, is exposed to UV light to cure the composition. A suitable UV light may be natural sunlight. Another suitable UV light may be a UV lamp, such as a 36-watt lamp commonly used in many nail salons. Such a UV lamp may operate at any wavelength required to cure the photopolymerizable composition, such as between 320 nm and 420 nm. The exposure time should be as long enough to allow for curing of the photopolymerizable composition. This exposure may be 5 seconds to 6 minutes.

The term “UV lamp” is meant to be interpreted broadly. It refers to any source of electromagnetic radiation that exhibits light in the 320 nm to 420 nm range at sufficient enough strength to cure the composition of the present invention. The term “UV lamp” includes traditional UV lamps that contain fluorescent lamps, such as compact fluorescent light bulbs, that give off UV light in the above-described ranges. The term “UV lamp” also refers to newer sources of light or UV radiation, such as light-emitting diode lamps (commonly referred to as “LED lamps”) that emit electromagnetic radiation which includes UV light in the 320 nm to 420 nm range at sufficient enough strength to cure the composition of the present invention. The term “UV lamp” also refers to any other type of source of light that comprises UV light in the 320 nm to 420 nm range at sufficient enough strength to cure the composition of the present invention.

The second method of forming an acrylic nail comprises the steps of contacting the photopolymerizable composition with a liquid to create a blend; placing the blend onto a nail; and exposing the blend to UV light.

The second method is similar to the first method, except that the photopolymerizable composition is not placed on the nail neat, but is first wetted with a liquid. According to the second method, the technician uses the liquid to help in lowering the viscosity of the composition.

The liquid of this method is selected from the group consisting of an acrylic nail monomer, solvent, oil, slip agent, and mixtures thereof. The solvent must be miscible with the photopolymerizable composition. The phrase “acrylic nail monomer” or the phrase “monomer liquid” means a liquid that is used by technicians to prepare acrylic nails.

The term “liquid” when referring to the monomer liquid means a liquid composition that is sufficiently homogeneous to be used for the preparation of acrylic nails. The liquid may be a uniform clear solution, a liquid that displays a Tyndall effect, a colloid, or a suspension in which any fine particles of the suspension do not precipitate during storage time. The liquid may be colorless, or it may be colored.

The third method of forming an acrylic nail comprises the steps of placing the photopolymerizable composition onto a nail; contacting the composition with a liquid; and exposing the mixture to UV light.

The third method is similar to the first method, except that the photopolymerizable composition is placed on the nail neat, and is then wetted with the liquid. According to the third method, the technician uses the liquid to help in lowering the viscosity of the composition after the composition is placed on the nail. The liquid of the third method is as described above for the second method.

The terms “cure”, “curing”, and like, are used herein are similar to those in the nail art, and are broadly encompassing terms. These terms refer to any portion of, or the entire process of, polymerization which is experienced under the UV light. A typical exemplary photopolymerizable composition for forming a cosmetic coating for nails according to various embodiments of the present invention comprises a continuous phase mixture and a discontinuous phase mixture. A typical continuous phase mixture comprises urethane (meth)acrylate oligomer, hydroxyalkyl (meth)acrylate, cycloalkyl (meth)acrylate, tri((meth)acrylate), a photoinitiator, an antioxidant and a colorant.

For each of the formulations, the above ingredients were added together and mixed until the composition was homogeneous. The order of addition of the ingredients did not show any appreciable differences in the performance of the composition.

A fractional factorial experiment was performed, wherein one factor was the identity of the discontinuous phase, and the wt % as another factor. The resulting viscosity, exotherm and qualitative assessment on the resulting composition were measured. Examples of several measurements are presented below.

| | Relative | Viscosity | |
|---|---|---|---|
| | Quantity | (cP at | Exotherm |
| Discontinuous Phase | (wt %) | 2 rps) | (° C.) |
| Polyurethane | 30 | 1,625,000 | 28.7 |
| Micronized polypropylene wax | 30-40 | 510,300 | 26.7 |
| Polymethylsilsesquioxane | 55 | 3,608,000 | 14.6 |

A sample bead of the above formulation was placed on a microscope slide. The bead had the viscosity and workability of a typical traditional two-part system about 4 minutes after the monomer liquid and the polymer have been mixed. The open time appeared to be much longer than typically necessary by even the most inexperienced nail technician. The bead was spread and exposed to a UV lamp, to yield a hard, durable substance.

### Viscosity Measurements

The viscosities of samples of the above formulation (prior to curing) were measured using a TA Instruments AR1500EX Rheometer using a cone and plate setup, with a 40 mm 1° steel cone. The temperature of the Peltier plate was set to 25° C., and the instrument was calibrated prior the use. The sample (0.5±0.05 g) was loaded in the center of the plate, the head was lowered to the truncation gap of 31 microns, and the sample was conditioned to 25° C. The measurements were taken under steady-state flow conditions, where the temperature and truncation gap were held constant throughout the run. The viscosity of the samples was measured at 5 points during the analysis, with each data point collected at a different, increasing shear rate. The initial shear rate is 2 s⁻¹ and each subsequent measurement was taken as the shear rate was increased by increments of 2 s⁻¹ until the final shear rate of 10 s⁻¹ was reached. The viscosity obtained at the 2 s⁻¹ shear rate was recorded as the final viscosity of the sample. The viscosity of a typical sample was between 500,000 to 5,000,000 centipoise.

### Exotherm Measurements

A 0.5 g sample of the above formulation was compared to a 0.5 g sample of a mixture of the same gel and a polymer powder, and the amount of photoinitiators was adjusted so that both samples had the same quantity of photoinitiators. The samples were applied to a thermocouple on a glass slide. After photoinitiation, there was a 19° C. rise in the sample without the polymer powder but only a 12° C. rise in the sample with the polymer powder.

### Stability Testing

Stability testing was performed one several samples of some formulations of continuous phase mixture and discontinuous phase mixture, including the examples above. Samples of each formulation were held at about 20° C. or at about 25° C. The samples were monitored at various time intervals and compared to one another to observe if any changes in physical state or performance of the samples could be noted; namely yellowing and polymerization. Any sample that remained relatively unchanged after 4 hours at such temperatures was said to pass stability testing.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

What is claimed is:

1. A photopolymerizable composition for forming a cosmetic coating for nails comprising:
(a) about 20 wt % to about 80 wt % of a discontinuous phase mixture comprised of one or more classes of particles with the mean particle size between about 1 micrometer and 100 micrometers, wherein the each class of particles is selected from the group consisting of a poly(C₁₋₁₂alkyl(meth)acrylate), a styrene, an aramid, a cellulose ester, an epoxy, a melamine-formaldehyde, a phenol-formadehyde, a poly(arylene sulfide), a poly(arylene terephthalamide), a polyacrylamide, a polyacrylonitrile, a polyalkylene, a polyamide, a polycarbonate, a polyester, a polyetheretherketone, a polyether, a polyether sulfone, a polyimide, a polylactam, a polylactone, a polyol, a polyphosphazene, a polysiloxane, a polyurea, a polyurethane, a polyvinyl alcohol, a polyvinyl halide, a polyvinyl acetate, a silicone, a wax, a rubber, a copolymer of any of the preceding, barium sulfate, boron nitrides, calcium sulfate, a ceramic, clay, diamond, glass, metal oxide, metal, mica, mineral, silicate, silicon dioxide, talc, titanium dioxide, an encapsulated version of any of the preceding, and a mixture thereof;
(b) 0 wt % to about 80 wt % of one or more (meth)acrylate oligomers;
(c) about 5 wt % to about 60 wt % of one or more (meth)acrylate monomers;
(d) one or more crosslinkers; and
(e) one or more photoinitiators;
wherein all wt % are with respect to the photopolymerizable composition.

2. The composition of claim 1, comprising about 40 wt % to about 60 wt % of the discontinuous phase mixture.

3. The composition of claim 2, wherein
the one or more (meth)acrylate monomers comprise a monomer selected from the group consisting of hydroxyalkyl (meth)acrylate, cycloalkyl (meth)acrylate, and a mixture thereof; and
the one or more (meth)acrylate oligomers comprise a urethane (meth)acrylate oligomer.

4. The composition of claim 1, wherein the discontinuous phase comprises a class of particles selected from the group consisting of a poly(C₁₋₁₂alkyl(meth)acrylate), a styrene, an aramid, a cellulose ester, an epoxy, a melamine-formaldehyde, a phenol-formaldehyde, a poly(arylene sulfide), a poly(arylene terephthalamide), a polyacrylamide, a polyacrylonitrile, a polyalkylene, a polyamide, a polycarbonate, a polyester, a polyetheretherketone, a polyether, a polyether sulfone, a polyimide, a polylactam, a polylactone, a polyol, a polyphosphazene, a polysiloxane, a polyurea, a polyurethane, a polyvinyl alcohol, a polyvinyl halide, a polyvinyl acetate, a silicone, a wax, a rubber, a copolymer of any of the preceding, an encapsulated version of any of the preceding, and a mixture thereof.

5. The composition of claim 1, wherein the discontinuous phase comprises a class of particles selected from the group consisting of a polyurea, a polyurethane, a copolymer of any of the preceding, an encapsulated version of any of the preceding, and a mixture thereof.

6. The composition of claim 1, wherein the discontinuous phase comprises a class of particles selected from the group consisting of barium sulfate, boron nitrides, calcium sulfate, a ceramic, clay, diamond, glass, metal oxide, metal, mica, mineral, silicate, silicon dioxide, talc, titanium dioxide, an encapsulated version of any of the preceding, and a mixture thereof.

7. The composition of claim 1, wherein the discontinuous phase comprises a class of particles that is metal oxide, wherein the metal comprises at least one transition metal.

8. The composition of claim 1, wherein the discontinuous phase comprises a class of particles that is metal oxide, wherein the metal comprises at least one transition metal and at least one main group element.

9. The composition of claim 1, wherein the discontinuous phase comprises a class of particles with the mean particle size is between about 5 micrometers and about 50 micrometers.

10. The composition of claim 1, wherein the discontinuous phase comprises a class of particles with the mean particle size is between about 5 micrometers and about 30 micrometers.

11. The composition of claim 1, wherein at least one of the one or more (meth)acrylate monomers comprise a (meth)acrylate selected from the group consisting of hydroxypropyl (meth)acrylate, hydroxyethyl (meth)acrylate, ethyl (meth)acrylate, propyl (meth)acrylate, butyl (meth)acrylate, isobutyl (meth)acrylate, sec-butyl (meth)acrylate, tetrahydrofurfuryl (meth)acrylate, caprolactone (meth)acrylate, (meth)acroyloxyethyl maleate, 2-hydroxyethyl (meth)acrylate/succinate, phthalic acid monoethyl (meth)acrylate, isobornyl (meth)acrylate, or a mixture thereof.

12. The composition of claim 1, wherein at least one of the one or more (meth)acrylate monomers comprises a hydroxyl-containing (meth)acrylate monomer.

13. The composition of claim 1, wherein the (meth)acrylate oligomer is selected from the group consisting of urethane (meth)acrylate, epoxy (meth)acrylate, epoxy urethane (meth)acrylate, (meth)acrylated acrylate, (meth)acrylated polyether, (meth)acrylated polycarbonate, (meth)acrylated cellulose, (meth)acrylated butadiene, (meth)acrylated styrene, polyester (meth)acrylate, polyester urethane (meth)acrylate, polyether urethane (meth)acrylate, polybutadiene urethane (meth)acrylate, and a mixture thereof.

14. The composition of claim 1, comprising about 20 wt % to about 80 wt % of one or more (meth)acrylate oligomers.

15. The composition of claim 1, comprising less than 1 wt % of one or more (meth)acrylate oligomers.

16. The composition of claim 1, wherein at least one of the one or more crosslinker is selected from the group consisting of trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, dipentaerythritol penta(meth)acrylate and ethoxylated isocyanuric acid tri(meth)acrylates.

17. The composition of claim 1, wherein at least one of the one or more crosslinkers is selected from the group consisting of tri(meth)acrylate, tetra(meth)acrylate, penta(meth)acrylate, and a mixture thereof.

18. The composition of claim 1, wherein at least one of the one or more photoinitiators is selected from the group consisting of 1-hydroxy-cyclohexylphenylketone; benzophenone; 2-benzyl-2-(dimethylamino)-1-(4-(4-morphorlinyl)phenyl)-1-butanone; 2,2-dimethoxy-2-phenyl acetophenone; 2-methyl-1-(4-methylthio)phenyl-2-(4-morphorlinyl)-1-propanone; 2,4,6-trimethylbenzoyldiphenyl-phosphine oxide; bis(2,4,6-trimethyl benzoyl)phenyl phosphine oxide; diphenyl-(2,4,6-trimethylbenzoyl)phosphine oxide; bis(2,6-dimethoxybenzoyl-2,4,4-trimethyl pentyl)phosphine oxide; 2-hydroxy-2-methyl-1-phenyl-1-propanone; phenyl bis(2,4,6-trimethylbenzoyl)phosphine oxide; benzyl-dimethylketal; isopropylthioxanthone; bis(η⁵-2,4-cyclopentadien-1-yl)bis[2,6-difluoro-3-(1H-pyrrol-1-yl)phenyl]titanium); α,α-dimethoxy α-phenyl acetophenone; ethyl (2,4,6-trimethyl benzoyl)phenyl phosphinate; phenyl (2,4,6-trimethyl benzoyl)phenyl phosphinate, methyl benzoyl formate, and mixtures thereof.

19. The composition of claim 1, wherein the composition further comprises a colorant or special effects pigment or a combination thereof.

20. The composition of claim 19, wherein the colorant is selected from the group consisting of ultramarine, manganese violet, zinc oxide, FD&C Blue No. 1, D&C Blue No. 4, Iron Blue, D&C Violet No. 2, and a mixture thereof.

21. The composition of claim 1, wherein the viscosity of the composition is greater than 400,000 centipoise at 25° C. as determined by a cone and plate rheometer using 40 mm 1° steel cone with the truncation gap of 31 microns operating at a 2 s⁻¹ shear rate on a 500 mg sample.

22. The composition of claim 1, wherein the viscosity of the composition is between about 500,000 centipoise and 5,000,000 centipoise at 25° C. as determined by a cone and plate rheometer using 40 mm 1° steel cone with the truncation gap of 31 microns operating at a 2 s⁻¹ shear rate on a 500 mg sample.

23. The composition of claim 1, wherein a 0.5-gram sample of the composition exhibits a temperature increase of less than about 30° C. during the exposure to UV light.

24. The composition of claim 1, wherein the composition is stable in a dark container at 49° C. for four months, or at 65° C. for 2 weeks.

25. The composition of claim 1, wherein the composition is stable in a dark container at less than 20° C. for four hours.

26. The composition of claim 1, wherein the composition is stable in a dark container at less than 15° C. for four hours.

27. A method of forming a cosmetic nail coating comprising the steps of:
(a) placing the composition of claim 1 onto a nail; and
(b) exposing the composition to actinic light.

28. A method of forming a cosmetic nail coating comprising the steps of:
(a) contacting the composition of claim 1 with a liquid selected from the group consisting of an acrylic nail monomer, solvent, oil, slip agent, photoinitiator and mixtures thereof to create a blend;
(b) placing the blend onto a nail; and
(c) exposing the blend to actinic light.

29. A method of forming a cosmetic nail coating comprising the steps of:
(a) placing the composition of claim 1 onto a nail;
(b) contacting the composition with a liquid selected from the group consisting of an acrylic nail monomer, solvent, oil, photoinitiator, and mixtures thereof; and
(c) exposing the mixture to actinic light.

30. A container containing the composition of claim 1, wherein the container is a tube container, a syringe container, or a foil packet, and wherein the container delivers a uniform bead of the composition.
